(19) United States
(12) Patent Application Publication
(10) Pub. No.: US 2014/0171466 A1
(43) Pub. Date: Jun. 19, 2014

(54) PAIN MANAGEMENT IN SICKLE CELL ANEMIA

(71) Applicants: Regents of the University of Minnesota, Minneapolis, MN (US); Astraea Therapeutics, LLC, Mountain View, CA (US)

(72) Inventors: Nurulain T. ZAVERI, Saratoga, CA (US); Kalpna GUPTA, Edina, CA (US)

(73) Assignees: Regents of the University of Minnesota, Minneapolis, MN (US); Astraea Therapeutics, LLC, Mountain View, CA (US)

(21) Appl. No.: 13/967,315

(22) Filed: Aug. 14, 2013

Related U.S. Application Data
(60) Provisional application No. 61/683,200, filed on Aug. 14, 2012.

(51) Int. Cl. C07D 401/04

(52) U.S. Cl. CPC C07D 401/04
USPC 514/323

(57) ABSTRACT

The present invention relates to a method for the management of pain associated with sickle cell disease. In particular, the method comprises administering to a patient in need of such pain management a therapeutically effective amount of a nociceptin (NOP) receptor agonist or an NOP receptor agonist/mu opioid receptor (MOR) partial agonist.

### STATEMENT AS TO FEDERALLY SPONSORED RESEARCH

This application was made with U.S. Government support under Grant Number R01DA014026, Grant Number 1R43H115984-01, and Grant Number R01DA027811, all awarded by the National Institutes of Health. The U.S. Government has certain rights in the invention.

### TECHNICAL FIELD

The present invention relates to a method for the management of pain associated with sickle cell disease. In particular, the method comprises administering to a patient in need of such pain management a therapeutically effective amount of a nociceptin (NOP) receptor agonist or an NOP receptor agonist/mu opioid receptor (MOR) partial agonist.

### BACKGROUND OF THE INVENTION

Sickle cell anemia is caused by an inherited mutation in the hemogloblin gene such that abnormal, sickle-shaped red blood cells are produced. These irregularly shaped cells become rigid and sticky, and slow or block blood flow and consequent oxygenation to parts of the body. No cure exists for people with sickle cell disease (SCD). SCD afflicts millions of people worldwide, and occurs in one of every 500 African-American and in one of every 1000 Hispanic children in the United States. In addition to hemolytic anemia, SCD is associated with unpredictable recurrent acute vaso-occlusive pain episodes (“crises”) and chronic pain. Pain starts early in life and continues to increase throughout life (Ballas, S. K., Hematology, Am. Soc. Hematol. Educ. Program, 2007, 97-105; NHLBI, The Management of Sickle Cell Disease, NIH, Bethesda, Md., 2002, Vol. 02-2117). Pain due to crises in SCD is considered to be more intense than labor pain. Recurrent episodes of crises and pain lead to increased hospitalization, poor quality of life, morbidity, and mortality in SCD.

Patients with SCD suffer with severe pain throughout life, which is challenging to treat. Opioids are the only widely used therapy, but unfortunately are associated with side effects such as constipation and development of tolerance and addiction. Higher doses of opioids are required to treat analogous pain in SCD as compared to other conditions due to increased systemic clearance of opioids in SCD patients (Darbari, D. S. et al., J. Pain, 2011, 12:531-8). Development of more effective analgesics to treat chronic and vaso-occlusive pain of human SCD therefore remains a critically unmet need. New approaches are critically required to treat pain and improve quality of life for patients with SCD.

### SUMMARY OF THE INVENTION

In one aspect, the present invention provides a method for the management of pain associated with sickle cell disease, comprising administering to a patient in need of such pain management a therapeutically effective amount of a nociceptin (NOP) receptor agonist, or a pharmaceutically acceptable salt, hydrate or solvate thereof. In one embodiment, the pain management comprises preventing or treating said pain. In another embodiment, the administering occurs during or after a sickle cell crisis. In a further embodiment, the NOP receptor agonist exhibits an NOP binding affinity of less than about 50 nM) and an agonist efficacy of about 50% to about 100% of nociceptin. In another embodiment, the agonist efficacy is determined in a GTP(γ)S assay.

In a further embodiment of the present invention, the NOP receptor agonist comprises a compound having the structure of Formula 1:

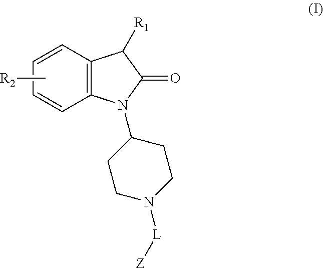

wherein:

R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;

R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;

Lis —(CHR³)_{P}—, —CH₂—Xᵥ—, or —C(═NH)—NR⁴;

pis an integer in the range of zero to 4 inclusive;

vis an integer in the range of 1 to 3 inclusive;

R³ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R³ may be the same or different;

R⁴ is selected from hydrogen, alkyl, cycloalkyl, cycloalkylalkyl, alkenyl, alkoxy, aryl and aralkyl;

X is independently CH₂, NR⁵ or 0, wherein at least one X, if v is greater than 1, is NR⁵ or O;

R⁵ is selected from hydrogen, alkyl, cycloalkyl, cycloalkylalkyl, alkenyl, alkoxy, aryl and aralkyl, and, in some preferred embodiments, when L is —(CHR³)_{P}—, p is 0; and

Z is hydrogen, cyclic hydrocarbyl, or arylcycloalkyl.

In one embodiment, Z has the structure:

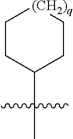

where q is an integer from 0-5.

In another aspect, the present invention provides a method for the management of pain associated with sickle cell disease, comprising administering to a patient in need of such pain management a therapeutically effective amount of a nociceptin (NOP) receptor agonist/mu opioid receptor (MOR) partial agonist, or a pharmaceutically acceptable salt, hydrate or solvate thereof. In one embodiment, the pain management comprises preventing or treating said pain. In another embodiment, the administering occurs during or after a sickle cell crisis. In a further embodiment, the NOP receptor agonist/MOR partial agonist exhibits an NOP and mu binding affinity (Kᵢ) of less than about 50 nM, an NOP agonist efficacy equal to about 50 to about 100% of that of nociceptin, and an mu efficacy equal to about 15% to about 75% of that of the peptide mu agonist DAMGO ([D-Ala2, N-MePhe4, Gly-ol]-enkephalin). In another embodiment, the NOP agonist efficacy is determined in an GTP(γ)S assay. In a further embodiment, the mu efficacy is determined in an GTP(γ)S assay.

In another embodiment of the present invention, the NOP receptor agonist/MOR partial agonist comprises a compound having the structure of Formula 1:

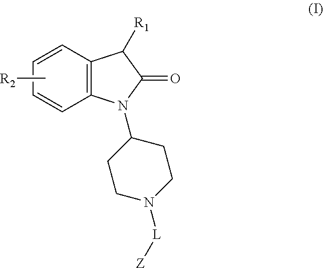

wherein:

R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;

R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;

L is —(CHR³)_{P}—, —CH₂—Xᵥ—, or —C(═NH)—NR⁴;

p is an integer in the range of zero to 4 inclusive;

v is an integer in the range of 1 to 3 inclusive;

R³ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R³ may be the same or different;

R⁴ is selected from hydrogen, alkyl, cycloalkyl, cycloalkylalkyl, alkenyl, alkoxy, aryl and aralkyl;

X is independently CH₂, NR⁵ or 0, wherein at least one X, if v is greater than 1, is NR⁵ or O;

R⁵ is selected from hydrogen, alkyl, cycloalkyl, cycloalkylalkyl, alkenyl, alkoxy, aryl and aralkyl, and, in some preferred embodiments, when L is —(CHR³)_{P}—, p is 0; and

Z is hydrogen, cyclic hydrocarbyl, or arylcycloalkyl.

In a further embodiment, Z has the structure:

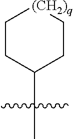

where q is an integer from 0-5.

In another aspect of the present invention, a pharmaceutical composition for managing pain associated with sickle cell disease is provided, comprising a therapeutically effective amount of an NOP agonist or an NOP agonist/mu opioid partial agonist, or a pharmaceutically acceptable salt, hydrate or solvate thereof, and a pharmaceutically acceptable carrier.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 demonstrates that the NOP receptor agonist AT-200 (Compound 3) reduces mechanical and deep tissue hyperalgesia in HbSS-BERK mice and their controls HbAA-BERK mice. Following baseline measurements for mechanical and deep tissue hyperalgesia, mice were injected with vehicle (2% dimethyl sulphoxide in 0.5% aq. hydroxypropylcellulose) or AT-200. Mechanical and deep tissue hyperalgesia was measured at the indicated times on the graph. (A-B) Mechanical threshold using von Frey filaments, measure of the cutaneous nociception is shown. Lower thresholds are indicative of increased sensitivity to cutaneous nociception. (C-D) Paw withdrawal frequency (PWF) in response to 10 applications of a 9.8 mN (1.0 g) von Frey monofilament in the same mice. A higher PWF indicates increased nociception. (E-F) Grip Force measurements indicating deep tissue/musculoskeletal pain are shown. Lower grip force suggests increased deep tissue hyperalgesia. Open squares with solid lines and star with dashed lines represent vehicle and AT-200 treatment in HbAA-BERK mice, respectively. Open triangles with solid lines and crosses with dashed lines represent vehicle and AT-200 treatment in HbSS-BERK mice, respectively. Data are shown as mean±SEM from 6 mice in HbAA-BERK mice per treatment and from 8 mice in HbSS-BERK mice per treatment. Statistical significance was calculated by comparing each value to BL (*) and between vehicle and AT-200 (†). *P<0.05 and **P<0.005 as compared to BL; and †P<0.05 and ††P<0.005 as compared to Vehicle for that time point. Mean age of mice±SEM in months were, HbAA-BERK Vehicle, 17.2±1.6, HbAA-BERK AT-200, 19.9±1.3, HbSS-BERK Vehicle, 21.2±1.2, and HbSS-BERK AT-200, 20.7±1.4. BL means baseline; 1 d means one day; Veh means vehicle.

FIG. 2 shows that the NOP receptor agonist AT-200 (Compound 3) reduces mechanical and deep tissue hyperalgesia following hypoxia-reoxygenation injury in HbSS-BERK mice and their controls HbAA-BERK mice. Following baseline measurements for mechanical and deep tissue hyperalgesia, mice were exposed to 3 h of hypoxia treatment and 1 h of reoxygenation at room air (H/R), and pain parameters were measured again. Mice were then injected with vehicle (2% dimethyl sulphoxide in 0.5% aq. hydroxypropylcellulose), morphine, or AT-200. Mechanical and deep tissue hyperalgesia was measured at the indicated times on the graph. (A-B) Cutaneous nociception was assessed using paw withdrawal threshold to von Frey filaments. Lower thresholds are indicative of increased sensitivity to cutaneous nociception. (C-D) Paw withdrawal frequency (PWF) in response to 10 applications of a 9.8 mN (1.0 g) von Frey monofilament in the same mice. A higher PWF indicates increased sensitivity to nociceptive stimuli. (E-F) Grip Force measurements indicating deep tissue/musculoskeletal pain are shown. Lower grip force suggests increased deep tissue hyperalgesia. Open squares with solid lines and star with dashed lines represent vehicle and AT-200 treatment in HbAA-BERK mice, respectively. Open triangles with solid lines, crosses with dashed lines, and solid circles with dashed lines represent vehicle, AT-200, and morphine treatment in HbSS-BERK mice, respectively. Data are shown as mean±SEM from 6 mice in HbAA-BERK mice per treatment and from 5-6 mice in HbSS-BERK mice per treatment. Statistical significance was calculated by comparing each value with BL (*) or with H/R (¶); and between vehicle and AT-200 (†). *P<0.05 and **P<0.005 as compared to BL; ¶P<0.05 and ¶¶P<0.005 as compared to H/R; and †P<0.05 and ††P<0.005 as compared to Vehicle for that time point. Mean age of mice±SEM in months were, HbAA-BERK Vehicle, 18.7±1.9, HbAA-BERK AT-200, 19.1±1.2, HbSS-BERK Vehicle, 20.9±1.7, and HbSS-BERK Compound 3, 21.2±1.7, and HbSS-BERK MS, 21.4±1.6. BL means baseline; H/R means hypoxia reoxygenation; 1 d means 1 day; Veh means vehicle; and MS means morphine.

FIG. 3 shows that the NOP receptor agonist AT-200 (Compound 3) reduces thermal hyperalgesia in HbSS-BERK mice and their controls HbAA-BERK mice. Following baseline measurements for mechanical and deep tissue hyperalgesia, mice were injected with vehicle (2% dimethyl sulphoxide in 0.5% aq. hydroxypropylcellulose) or AT-200. Thermal hyperalgesia was measured at the indicated times on the graph. (A-B) Paw Withdrawal latency (PWL) after application of a cold stimulus (cold plate at 4±1° C.) (A-B) and heat stimulus (E-F) were recorded for each mouse. Shorter PWL (in seconds) in response to heat stimulus is indicative of increased thermal hyperalgesia. A higher PWF in a 2-minute period and lower PWL on a cold plate are indicative of increased sensitivity to cold induced nociception. Open squares with solid lines and star with dashed lines represent vehicle and AT-200 treatment in HbAA-BERK mice, respectively. Open triangles with solid lines and crosses with dashed lines represent vehicle and AT-200 treatment in HbSS-BERK mice, respectively. Data are shown as mean±SEM from 6 mice in HbAA-BERK mice per treatment and from 8 mice in HbSS-BERK mice per treatment. Statistical significance was calculated by comparing each value to BL (*) and between vehicle and AT-200 (†). *P<0.05 and **P<0.005 as compared to BL; and †P<0.05 and ††P<0.005 as compared to Vehicle for that time point. Mean age of mice±SEM in months were, HbAA-BERK Vehicle, 17.2±1.6, HbAA-BERK AT-200, 19.9±1.3, HbSS-BERK Vehicle, 21.2±1.2, and HbSS-BERK AT-200, 20.7±1.4. BL means baseline; 1 d means 1 day; and Veh means vehicle.

FIG. 4 shows that the NOP receptor agonist AT-200 (Compound 3) reduces thermal hyperalgesia following hypoxia reoxygenation in HbSS-BERK mice and their controls HbAA-BERK mice. Following baseline measurements of thermal hyperalgesia, mice were exposed to 3 h of hypoxia treatment and 1 h of reoxygenation at room air (H/R), and pain parameters were measured again. Mice were then injected with vehicle (2% dimethyl sulphoxide in 0.5% aq. hydroxypropylcellulose), morphine, or AT-200. Mechanical and deep tissue hyperalgesia was measured at the indicated times on the graph. (A-B) Paw Withdrawal latency (PWL) after application of a cold stimulus (cold plate at 4±1° C.) (A-B) and heat stimulus (E-F) were recorded for each mouse. Shorter PWL (in seconds) in response to heat stimulus is indicative of increased thermal hyperalgesia. A higher PWF in a 2-minute period and lower PWL on a cold plate are indicative of increased sensitivity to cold induced nociception. Open squares with solid lines and star with dashed lines represent vehicle and AT-200 treatment in HbAA-BERK mice, respectively. Open triangles with solid lines, crosses with dashed lines, and solid circles with dashed lines represent vehicle, AT-200, and morphine treatment in HbSS-BERK mice, respectively. Data are shown as mean±SEM from 6 mice in HbAA-BERK mice per treatment and from 5-6 mice in HbSS-BERK mice per treatment. Statistical significance was calculated by comparing each value with BL (*) or with H/R (¶); and between vehicle and AT-200 (†). *P<0.05 and **P<0.005 as compared to BL; ¶ P<0.05 and ¶¶P<0.005 as compared to H/R; and †P<0.05 and ††P<0.005 as compared to Vehicle for that time point. Mean age of mice SEM in months were, HbAA-BERK Vehicle, 18.7±1.9, HbAA-BERK AT-200, 19.1±1.2, HbSS-BERK Vehicle, 20.9±1.7, and HbSS-BERK AT-200, 21.2±1.7, and HbSS-BERK MS, 21.4±1.6. BL means baseline; H/R means hypoxia reoxygenation; 1 d means 1 day; Veh means vehicle; and MS means morphine.

FIG. 5 shows that the NOP receptor agonist AT-200 (Compound 3) does not affect cutaneous blood flow measured by laser doppler velocimetry in HbSS-BERK mice and their controls HbAA-BERK mice. Following baseline measurements for blood flow, mice were injected with vehicle (2% dimethyl sulphoxide in 0.5% aq. hydroxypropylcellulose) or AT-200. Blood flow was measured at the indicated times on the graph. (A-B) HbSS-BERK show increased vascular flow measured by laser doppler velocimetry. Open squares with solid lines and star with dashed lines represent vehicle and AT-200 treatment in HbAA-BERK mice, respectively. Open triangles with solid lines and crosses with dashed lines represent vehicle and AT-200 treatment in HbSS-BERK mice, respectively. Data are shown as mean±SEM from 6 mice in HbAA-BERK mice per treatment and from 8 mice in HbSS-BERK mice per treatment. Statistical significance was calculated by comparing each value to BL (*) and between vehicle and AT-200 (†). *P<0.05 and **P<0.005 as compared to BL; and †P<0.05 and ††P<0.005 as compared to Vehicle for that time point. Mean age of mice±SEM in months were, HbAA-BERK Vehicle, 17.2±1.6, HbAA-BERK AT-200, 19.9±1.3, HbSS-BERK Vehicle, 21.2±1.2, and HbSS-BERK AT-200, 20.7±1.4. BL means baseline; 1 d means 1 day; Veh means vehicle.

FIG. 6 demonstrates that the NOP receptor agonist AT-200 (Compound 3) reduces cutaneous blood flow following hypoxia-reoxygenation injury in HbSS-BERK mice and their controls HbAA-BERK mice. Following baseline measurements for blood flow, mice were exposed to 3 h of hypoxia treatment and 1 h of reoxygenation at room air (H/R), and blood flow was measured again. Mice were then injected with vehicle (2% dimethyl sulphoxide in 0.5% aq. hydroxypropylcellulose), morphine, or AT-200. Blood flow was measured at the indicated times on the graph. (A-B) HbSS-BERK show increased vascular flow measured by laser doppler velocimetry. Open squares with solid lines and star with dashed lines represent vehicle and AT-200 treatment in HbAA-BERK mice, respectively. Open triangles with solid lines, crosses with dashed lines, and solid circles with dashed lines represent vehicle, AT-200, and morphine treatment in HbSS-BERK mice, respectively. Data are shown as mean±SEM from 6 mice in HbAA-BERK mice per treatment and from 5-6 mice in HbSS-BERK mice per treatment. Statistical significance was calculated by comparing each value with BL (*) or with H/R (¶); and between vehicle and AT-200 (†). *P<0.05 and **P<0.005 as compared to BL; ¶P<0.05 and ¶¶P<0.005 as compared to H/R; and †P<0.05 and ††P<0.005 as compared to Vehicle for that time point. Mean age of mice±SEM in months were, HbAA-BERK Vehicle, 18.7±1.9, HbAA-BERK AT-200, 19.1±1.2, HbSS-BERK Vehicle, 20.9±1.7, and HbSS-BERK AT-200, 21.2±1.7, and HbSS-BERK MS, 21.4±1.6. BL means baseline; H/R means hypoxia reoxygenation; 1 d means 1 day; Veh means vehicle; and MS means morphine.

FIG. 7 demonstrates that The NOP receptor agonist AT-200 (Compound 3) reduces dorsal skin temperature measured by infrared thermography in HbSS-BERK mice. Following baseline measurements for blood flow, mice were injected with vehicle (2% dimethyl sulphoxide in 0.5% aq. hydroxypropylcellulose) or AT-200. Skin temperature was measured at the indicated times on the graph. (A-B) HbSS-BERK show decreased skin temperature measured by infrared thermography. Open squares with solid lines and star with dashed lines represent vehicle and AT-200 treatment in HbAA-BERK mice, respectively. Open triangles with solid lines and crosses with dashed lines represent vehicle and AT-200 treatment in HbSS-BERK mice, respectively. Data are shown as mean±SEM from 6 mice in HbAA-BERK mice per treatment and from 8 mice in HbSS-BERK mice per treatment. Statistical significance was calculated by comparing each value to BL (*) and between vehicle and AT-200 (†). *P<0.05 and **P<0.005 as compared to BL; and †P<0.05 and ††P<0.005 as compared to Vehicle for that time point. Mean age of mice±SEM in months were, HbAA-BERK Vehicle, 17.2±1.6, HbAA-BERK AT-200, 19.9±1.3, HbSS-BERK Vehicle, 21.2±1.2, and HbSS-BERK AT-200, 20.7±1.4. BL means baseline; 1 d means 1 day; and Veh means vehicle.

FIG. 8 demonstrates that the NOP receptor agonist AT-200 (Compound 3) reduces dorsal skin temperature measured by infrared thermography following hypoxia-reoxygenation injury in HbSS-BERK mice and their controls HbAA-BERK mice. Following baseline measurements for blood flow, mice were exposed to 3 h of hypoxia treatment and 1 h of reoxygenation at room air (H/R), and blood flow was measured again. Mice were then injected with vehicle (2% dimethyl sulphoxide in 0.5% aq. hydroxypropylcellulose), morphine, or AT-200. Temperature was measured at the indicated times on the graph. (A-B) HbSS-BERK show increased skin temperature measured by infrared thermography. Open squares with solid lines and star with dashed lines represent vehicle and AT-200 treatment in HbAA-BERK mice, respectively. Open triangles with solid lines, crosses with dashed lines, and solid circles with dashed lines represent vehicle, AT-200, and morphine treatment in HbSS-BERK mice, respectively. Data are shown as mean±SEM from 6 mice in HbAA-BERK mice per treatment and from 5-6 mice in HbSS-BERK mice per treatment. Statistical significance was calculated by comparing each value with BL (*) or with H/R (¶); and between vehicle and AT-200(†). *P<0.05 and **P<0.005 as compared to BL; ¶P<0.05 and ¶¶P<0.005 as compared to H/R; and †P<0.05 and ††P<0.005 as compared to Vehicle for that time point. Mean age of mice±SEM in months were, HbAA-BERK Vehicle, 18.7±1.9, HbAA-BERK AT-200, 19.1±1.2, HbSS-BERK Vehicle, 20.9±1.7, and HbSS-BERK AT-200, 21.2±1.7, and HbSS-BERK MS, 21.4±1.6. BL means baseline; H/R means hypoxia reoxygenation; 1 d means 1 day; Veh means vehicle; and MS means morphine.

FIG. 9 shows the development of analgesic tolerance in an acute pain tail-flick assay in mice. NOP/mu bifunctional agonist Compound 4 (1 mg/kg) maintains its tail-flick latency (closed squares) after 10 days of daily dosing, compared to morphine at both 1 and 3 mg/kg (open and closed diamonds).

FIG. 10 demonstrates the results from an anti-nociceptive (tail-flick assay) and anti-allodynic (von Frey) activity evaluation in the mouse spinal nerve ligation model. (A, B): NOP agonist/mu agonist Compound 3 results; (C, D): NOP agonist Compound 1; (A, C): Tail flick latency. Morphine positive control. (B, D): Anti-allodynic activity using Von Frey filaments. Data are means (±S.E.M.)*, significant difference from vehicle controls; +, significant difference from morphine control.

FIG. 11 shows the structures of Compounds 1-6.

### DETAILED DESCRIPTION OF THE INVENTION

### Definitions and Nomenclature

In this specification and in the claims which follow, reference will be made to a number of terms which shall be defined to have the following meanings.

The term “alkaryl” refers to an aryl group with an alkyl substituent, and the term “aralkyl” refers to an alkyl group with an aryl substituent, wherein “aryl” and “alkyl” are as defined above. Preferred aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, preferably 2 to 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁₋₆ alkoxy” or “lower alkoxy” herein contain 1 to 3 carbon atoms, and particularly preferred such substituents contain 1 or 2 carbon atoms (i.e., methoxy and ethoxy).

The term “alkyl” as used herein refers to a branched or unbranched saturated hydrocarbon group typically. although not necessarily, containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred substituents identified as “C₁₋₆ alkyl” or “lower alkyl” contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkylidene” refers to a linear or branched alkylidene group containing 1 to about 6 carbon atoms, such as methylene, ethylidene, propylidene, isopropylidene, butylidene, and the like.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 20 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “arylcycloalkyl” as used herein, and unless otherwise specified, refers to an aromatic substituent containing a single aromatic ring or multiple aromatic rings that are fused together with one or more cycloalkyl groups. The aromatic and the cycloalkyl rings may be substituted or unsubstituted. Preferably, the aromatic rings contain five to six carbon atoms. Preferably, the cycloalkyl groups contain five to eight carbon atoms. Examples include indanyl; benzocyclohexanyl; benzocycloheptanyl, 16,17-dihydro-15H-cyclopenta[a]phenanthrenyl; and 2,3,3a,4,5,6-hexahydro-1H-phenalen-1-yl.

The term “aryloxy” as used herein refers to an aryl group bound through a single, terminal ether linkage, wherein “aryl” is as defined above. An “aryloxy” group may be represented as −0-aryl where aryl is as defined above. Preferred aryloxy groups contain 5 to 20 carbon atoms, and particularly preferred aryloxy groups contain 5 to 14 carbon atoms. Examples of aryloxy groups include, without limitation, phenoxy, a-halo-phenoxy, m-halo-phenoxy, p-halophenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “cyclic” refers to an alicyclic or aromatic substituent, group, or Compound that may or may not be substituted and/or heteroatom containing, and that may be monocyclic, bicyclic, or polycyclic.

The terms “halo” is used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including unsubstituted, substituted, non-heteroatom-containing and heteroatom-containing hydrocarbyl moieties.

The term “saturated” is intended to include both fully saturated compounds, in particular ring structures such as cycloalkyl groups, as well as partially saturated compounds such as cycloalkenyl groups. For example, tetrahydronaphthyl is a partially saturated ring system and therefore is considered a “saturated” ring system herein. The term “unsaturated” refers to fully unsaturated moieties.

By “substituted” as in “substituted hydrocarbyl” and the like, as alluded to in some of the aforementioned definitions, is meant that in the hydrocarbyl or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁₋₂₄ alkoxy, C₂₋₂₄ alkenyloxy, C₂₋₂₄ alkynyloxy, C₅₋₂₀ aryloxy, acyl (including C₂₋₂₄ alkylcarbonyl (—CO-alkyl) and C₆₋₂₀ arylcarbonyl (—CO-aryl)), acyloxy (—O-acyl), C₂₋₂₄ alkoxycarbonyl (—(CO)—O-alkyl), C₆₋₂₀ aryloxycarbonyl (—(CO)—O-aryl), halocarbonyl (—CO)—X where X is halo), C₂₋₂₄ alkylcarbonato (—O—(CO)—O-alkyl), C₆₋₂₀ arylcarbonato (—O—(CO)—O-aryl), carboxy (—COOH), carboxylate (—COO), carbamoyl (—(CO)—NH₂), mono (C₁₋₂₄ alkyl)-substituted carbamoyl (—(CO)—NH(C₁₋₂₄ alkyl)), di-(C₁₋₂₄ alkyl)-substituted carbamoyl (—(CO)N(C₁₋₂₄ alkyl)₂), mono-substituted arylcarbamoyl (—(CO)—NH-aryl), thiocarbamoyl (—(CS)—NH₂), carbamido (—NH—(CO)—NH₂), cyano (—C═N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁₋₂₄ alkyl)-substituted amino, mono- and di-(C₅₋₂₀ aryl)-substituted amino, C₂₋₂₄ alkylamido (—NH—(CO)-alkyl), C₆₋₂₀ arylamido (—NH—(CO)aryl), imino (—CR═NH where R is hydrogen, C₁₋₂₄ alkyl, C₅₋₂₀ aryl, C₆₋₂₄ alkaryl, C₆₋₂₄ aralkyl, etc.), alkylimino (—R═N(alkyl), where R=hydrogen, alkyl, aryl, alkaryl, etc.), arylimino (—CR═N(aryl), where R is hydrogen, alkyl, aryl, alkaryl, etc.), nitro (—NO₂), nitroso (—NO), sulfa (—SO₂-0H-1), sulfonato (—SO₂—O—), C₁₋₂₄ alkylsulfanyl (—S-alkyl; also termed “alkylthio”), arylsulfanyl (—S-aryl; also termed “arylthio”), C₁₋₂₄ alkylsulfinyl (—(SO)-alkyl), C₅₋₂₀ arylsulfinyl (—(SO)-aryl), C₁₋₂₄ alkylsulfonyl (—SO₂-alkyl), C₅₋₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphine (—PH₂); and the hydrocarbyl moieties C₁₋₂₄ alkyl (preferably C₁₋₁₈ alkyl, more preferably C₁₋₁₂ alkyl, most preferably C₁₋₆-alkyl), C₂₋₂₄ alkenyl (preferably C₂₋₁₈ alkenyl, more preferably C₂₋₁₂ alkenyl, most preferably C₂₋₆ alkenyl), C₂₋₂₄ alkynyl (preferably C₂-18 alkynyl, more preferably C₂₋₁₂ alkynyl, most preferably C₂₋₆ alkynyl), C₅₋₂₀ aryl (preferably C₅₋₁₄ aryl), C₆₋₂₄ alkaryl (preferably C₆₋₁₈ alkaryl), and C₆₋₂₄ aralkyl (preferably C₆₋₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present. Similarly, the phrase an “optionally present” double bond as indicated by a dotted line (- - - ) in the chemical formulae herein means that a double bond may or may not be present, and, if absent, a single bond is indicated.

When referring to a Compound of the invention, applicants intend the term “compound” to encompass riot only the specified molecular entity but also its pharmaceutically acceptable, pharmacologically active equivalents, including, but not limited to, salts, esters, amides, prodrugs, conjugates, active metabolites, and other such derivatives, analogs, and related compounds.

### INTRODUCTION

The present invention relates to a method for the management of pain associated with sickle cell disease (SCD), comprising administering to a patient in need of such pain management a therapeutically effective amount of a nociceptin (NOP) receptor agonist or an NOP receptor agonist/mu opioid receptor (MOR) partial agonist, or a pharmaceutically acceptable salt, hydrate or solvate thereof. Modulation of SCD-related pain includes preventing such pain by administering a composition of the present invention before a sickle cell crisis occurs or administering the composition during or after a sickle cell crisis.

Morphine and its congeners provide a sub-optimal approach to treat pain in sickle cell disease (SCD) due to side effects, risk of addiction, and development of tolerance. The liabilities associated with use of morphine and mu opioid receptor (MOR) agonist analgesics may be avoided by using nociceptin receptor agonists or nociceptin receptor agonist/mu opioid receptor partial agonists of the invention to treat or prevent SCD pain. The nociceptin receptor (NOPR) and its endogenous ligand nociceptin/orphanin FQ (N/OFQ) belonging to the opioid receptor (OR) and opioid family respectively, are involved in nociceptive signaling. Small molecule NOPR agonists are non-addicting, while providing analgesia in acute and chronic pain models.

### Sickle Cell Disease

SCD is an autosomal recessive disorder caused by substitution of a valine residue for glutamic acid at position 6 (or Adenine to Thymidine transversion on codon 6) in the beta-globin gene, resulting in sickle hemoglobin (HbS) (Rees, D. C., et al., “Sickle-cell disease”, Lancet, 2011, 376:2018-31j). Under low oxygen, HbS polymerizes to form rigid fibers and confers a sickle shape to the red blood cells (RBCS). Sickle RBC exhibit reduced flexibility resulting in erythrocyte membrane damage and hemolysis. Sickle RBCs cluster together, occluding the blood vessels and impairing oxygen supply to the limbs and other organs, resulting in cumulative organ damage and acute painful SCD crises episodes. Thus, SCD is associated both with unpredictable, recurrent acute vaso-occlusive pain episodes (crises) and chronic pain (Ballas, 2007; NHLBI, 2002; Dampier, C., et al., J. Pain, 2002, 3:461-70; Smith, W. R., Expert Rev. Hematol., 2010, 4:237-9).

The pathobiology of pain in SCD remains understudied and treatment choices remain a major challenge. Opioid treatment, the mainstay of therapy, remains a suboptimal approach due to side effects, tolerance, addiction and altered clearance of opioids in SCD (Kohli, 2010; Dampier, C. D., et al., J. Pediatr., 1995, 126:461-7). Pain starts early in life and continues to increase with age, thus requiring recurrent use of opioids. But the consequent side effects of opioid therapy (constipation, tolerance, dependence) lead to sub-optimal analgesia, poor quality of life and shorter life-span (Ballas, 2007; Smith, 2010). Additional pain therapies, preferably with different pharmacological targets, are critically required to treat pain in SCD.

Mechanism(s) underlying pain in SCD remain unknown. Kalpna Gupta's laboratory was the first to initiate studies on the mechanism(s) of pain in SCD using transgenic mouse models that express human sickle hemoglobin (Kohli, D. R., et al., Blood, 2010, 116:456-65; Cain, D. M., et al., Brit. J. Haematol., 2012, 156(4):535-44). These mouse models exhibit the hematologic disease, organ pathology and features of pain observed in human SCD. The Gupta laboratory demonstrated that HbSS-BERK sickle mice expressing>99% human sickle hemoglobin exhibit tonic hyperalgesia similar to chronic pain in SCD4 and that severe pain due to crises can be modeled in these mice by inciting hypoxia-reperfusion injury but not in control mice expressing normal human hemoglobin (Kohli, D. R., et al., Blood, 2010, 116:456-65). These studies also showed that that deep tissue pain, analyzed by measuring “grip force”, is a distinguishing feature of pain in SCD as compared to other conditions. Notably, only high dose (20 mg/kg) morphine was effective in treating tonic- and CFA-induced-hyperalgesia in these mice, thus warranting the discovery of new, improved analgesics to treat pain in SCD.

The Gupta laboratory performed an examination of chronic pain, neurochemical changes and analgesic effects of morphine and cannabinoids in transgenic sickle mice (Kohli, et al., 2010). Paw withdrawal threshold (PWT) and withdrawal latency (PWL) to mechanical and thermal stimuli, respectively, and grip force were lower in homozygous Berkley (BERK) sickle mice compared to control mice expressing normal human hemoglobin A (HbA-BERK). These results suggest that chronic deep/musculoskeletal and cutaneous hyperalgesia is occurring, similar to the pain features in patients with SCD. Peripheral nerves and blood vessels were structurally altered in BERK skin, with decreased expression of the mu opioid receptor (MOR). Reduced expression of MOR may contribute to the need for high doses of morphine to treat pain in SCD. Hyperalgesia was markedly attenuated by high dose morphine (20 mg/kg), but not by relatively lower dose (10 mg/kg). However, a low dose of the cannabinoid receptor agonist CP 55,940 (0.3 mg/kg) attenuated hyperalgesia (Kohli, et al., 2010), indicating the robustness of this model of SCD to test newer analgesics.

Transgenic mouse models can also simulate pain due to SCD crises by inflicting acute pain according to techniques known in the art. For example, such transgenic mouse models with variable expression of sickle hemoglobin may be used to test for pain reduction and include NY1 DD, S+S^{Antilles} and BERK expressing ˜23%, ˜78% and >99% sickle hemoglobin, respectively (Cain, D. M., et al., Brit. J. Haematol., 2012, 156(4):535-44). Young (≦3 month old) NY1 DD and S+S^{Antilles} mice (having modest and moderate sickle phenotype, respectively) and BERK exhibit evidence of deep tissue/musculoskeletal pain. Cain, et al., reported that deep tissue pain and cold sensitivity in S+S^{Antilles} mice increased significantly with both age and incitement of hypoxia/reoxygenation (H/R) (2012). C57/BL6 mice (genetic background strain of NY1 DD and S+S^{Antilles}) were hypersensitive to mechanical and heat stimuli. H/R treatment of BERK mice with resulted in significantly decreased withdrawal thresholds and enhanced mechanical, thermal and deep tissue hyperalgesia. Deep hyperalgesia incited by H/R in BERK was ameliorated by CP 55940, a cannabinoid receptor agonist. Thus, assessment of deep tissue pain appears to be the most sensitive measure to study sickle cell pain, and BERK mice may best model the vaso-occlusive pain of SCD.

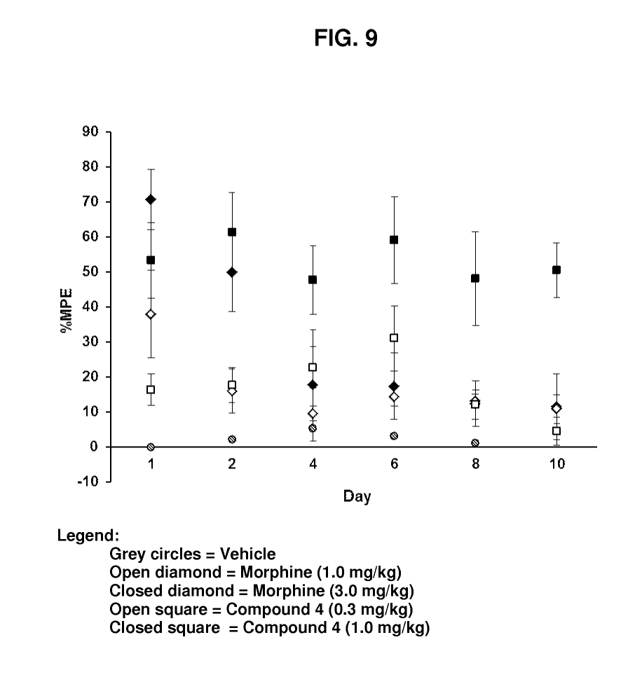

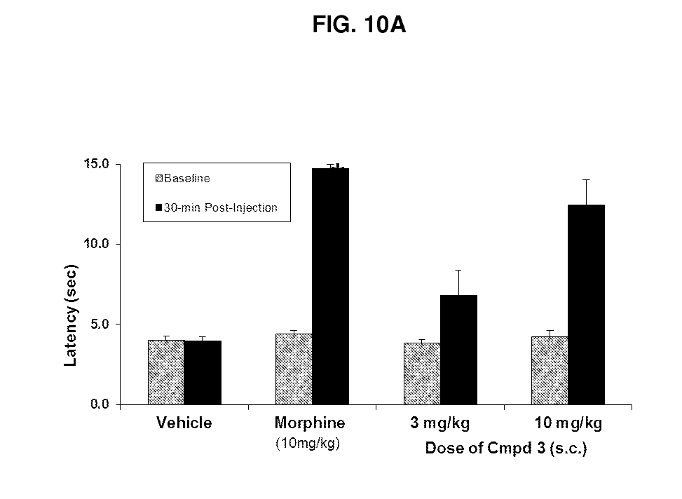

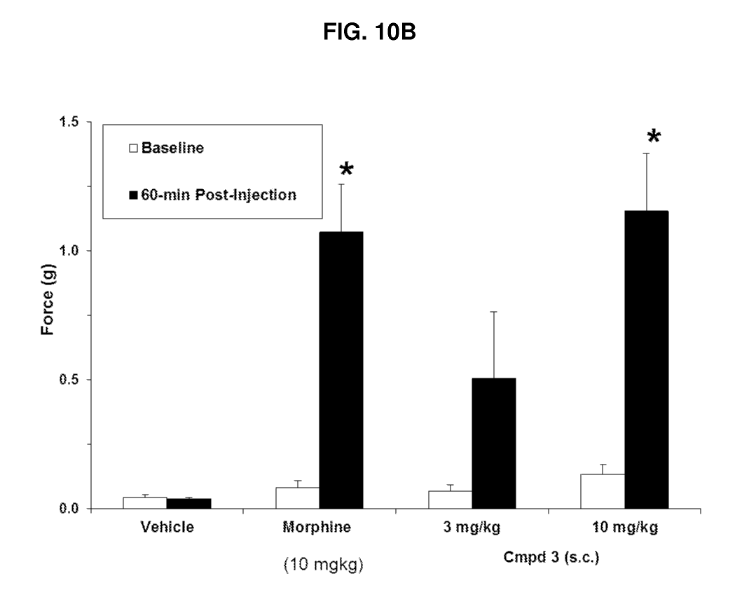

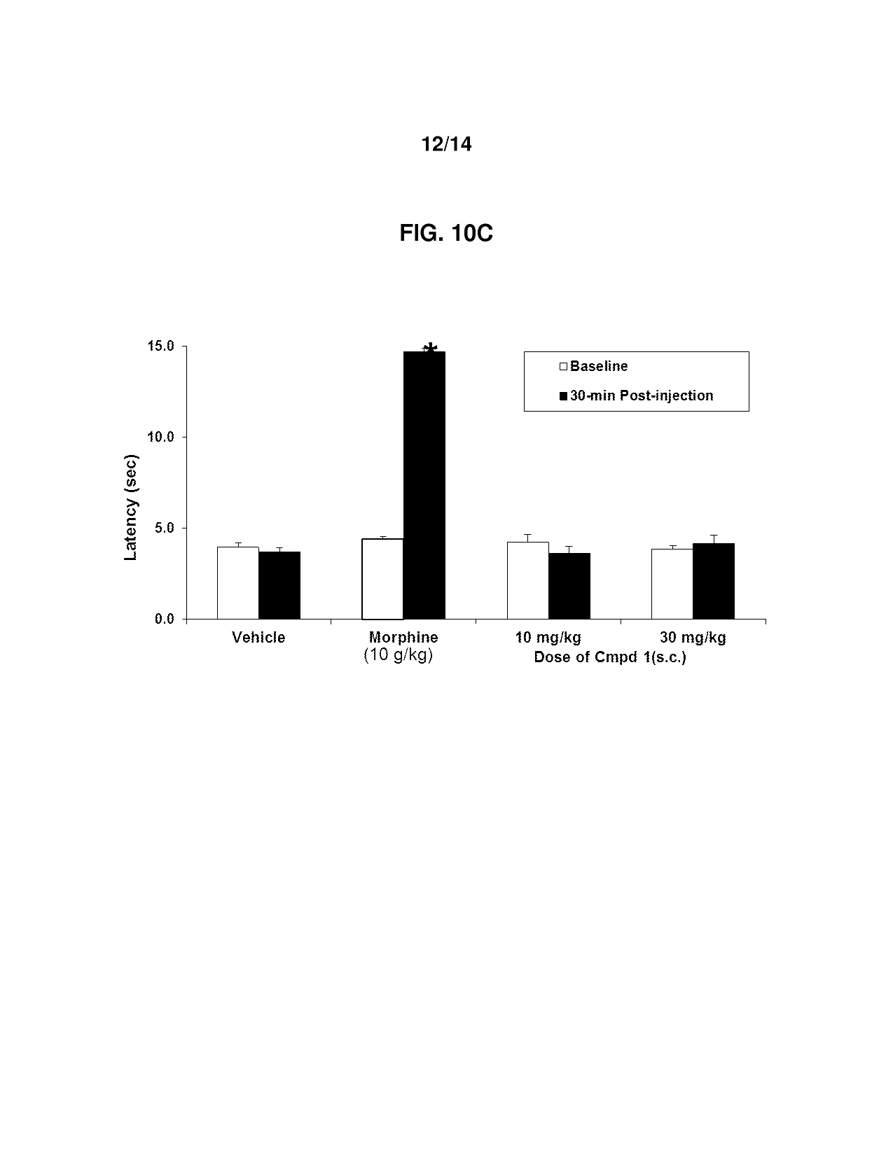

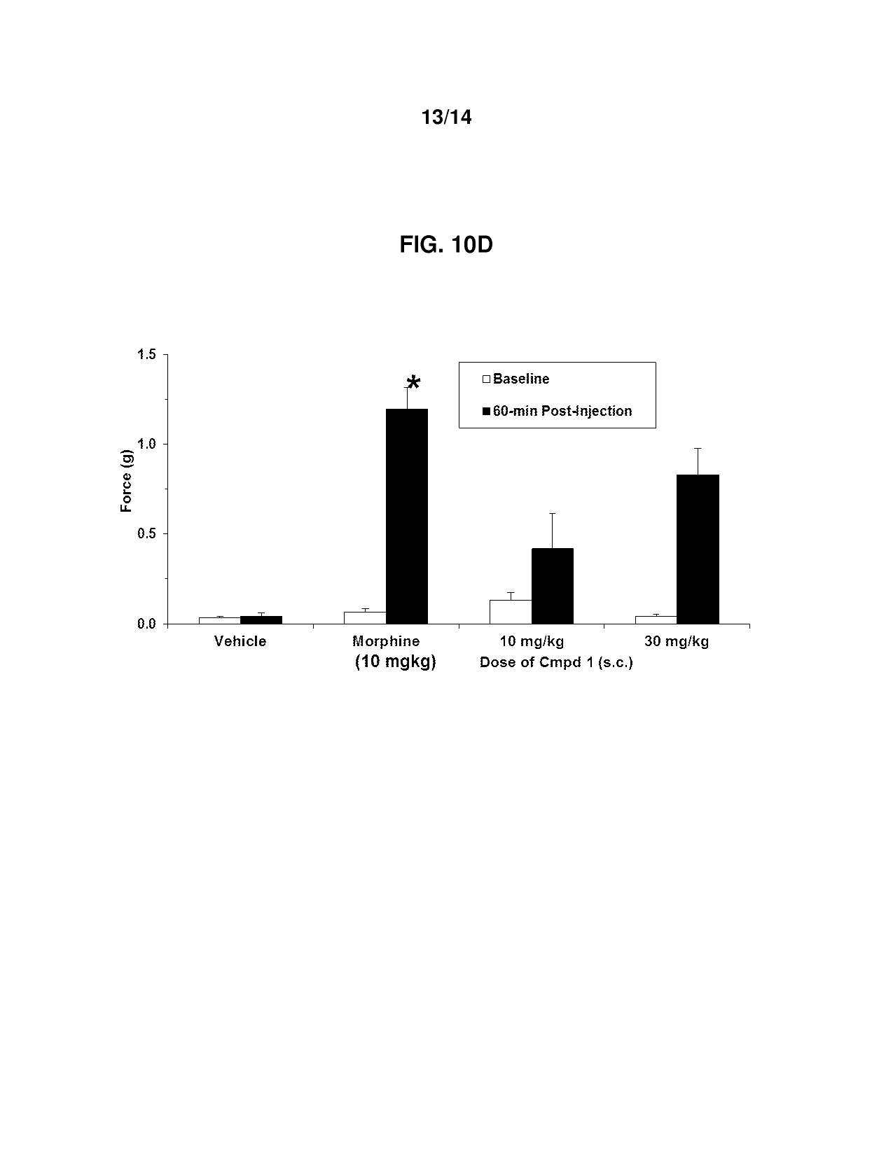

Of particular relevance with respect to new targets for pain therapeutics is the nociceptin receptor (NOP), the fourth member of the opioid receptor family, and its endogenous peptide ligand, nociceptin/orphanin FQ (N/OFQ). The NOP receptor and N/OFQ are found throughout the brain and spinal cord particularly in pain-processing pathways (Darland, T.; Grandy, D. K., Br. J. Anaesth., 1998, 81:29-37), and modulate opioid function by blocking opioid reward and tolerance. Zaveri and co-workers have determined that small-molecule NOP agonists have anti-nociceptive and anti-allodynic activity in animal models of chronic pain (Khroyan, T. V., et al., J. Pharmacol Exp. Ther., 2011, 339:687-693), whereas bifunctional NOP agonists with mu opioid partial agonist activity show potent analgesic effects on acute and chronic pain, are mainly non-rewarding (Toll, L., et al., J. Pharmacol Exp. Ther., 2009, 331:954-964), and do not develop tolerance upon continued administration (see FIG. 9). NOP agonism provides a promising approach for pain treatment devoid of addictive properties and side-effects.

Because they are in the opioid family, both the NOP receptor and its endogenous ligand N/OFQ have been extensively investigated for their effects on nociception and on opioid-mediated pharmacology such as reward and tolerance (Lambert, D. G., Nat. Rev. Drug Discov., 2008, 7:694-710). The physiological actions of the endogenous ligand N/OFQ, particularly with respect to nociceptive effects, appear to be region- and assay-specific. At the supraspinal level, N/OFQ appears to be pro-nociceptive when injected intracerebroventricularly (i.c.v). However, N/OFQ is analgesic when injected intrathecally (i.t.) in both acute and chronic models of pain (Tian, J. H., et al., Br. J. Pharmacol, 1997, 120:676-80; Yamamoto, T., et al., Brain Res., 1997, 754:329-32; Courteix, C., et al., Pain, 2004, 110:236-45). In addition to its involvement in pain pathways, N/OFQ's modulation of opioid effects is evident in a variety of paradigms. N/OFQ injected i.c.v. can attenuate development of morphine tolerance (Lutfy, K., et al., Br. J. Pharmacol, 2001, 134:529-34).

Within the reward circuitry, i.c.v. N/OFQ suppresses basal and morphine-stimulated dopamine (DA) release in the nucleus accumbens (Di Giannuario, A., et al., Peptides, 2000, 21:125-30; Di Giannuario, et al., Neurosci. Lett., 1999, 272:183-6; Murphy, N. P., et al., Neuroscience, 1996, 75:1-4; Murphy, N. P., et al., J. Neurochem., 1999, 73:179-86), suggesting the utility of N/OFQ and NOP agonists in reducing the rewarding effects of opioids and other drugs of abuse. Consistent with its inhibition of DA release, N/OFQ has been shown to block morphine and cocaine place preference (CPP), as well as self administration of alcohol (Ciccocioppo, R., et al., Psychopharmacology (Berl), 1999, 141:220-4; Murphy, N. P., et al., Brain Res., 1999, 832:168-70; Ciccocioppo, R., et al., Eur. J. Pharmacol., 2000, 404:153-9; Maidment, N. T., et al., Neuroreport, 2002, 13:1137-40; Koizumi, M., et al., J. Neurochem., 2004, 89:257-63). These effects are also evident with systemically administered NOP agonists, such as Ro 64-6198 (Shoblock, J. R., et al., Neuropharmacology, 2005, 49:439-46), which block morphine conditioned place preference (CPP) in mice. Therefore, it is now well accepted that supraspinally, N/OFQ has an anti-opioid function and can modulate (block) opioid-induced tolerance and reward. In contrast, when given spinally, N/OFQ produces an opioid-like anti-nociceptive effect (Zeilhofer, H. U.; Calo, G., J. Pharmacol Exp. Ther., 2003, 306:423-9).

The spinal anti-nociceptive action of N/OFQ is dose dependent, naloxone insensitive, and is mimicked by the NOP-selective peptide agonist UFP-112 (Rizzi, A., et al., Peptides, 2007, 28:1240-1251) and reversed by selective NOP antagonist SB-612111. This spinal anti-nociceptive action of NOP agonists has been confirmed in nonhuman primates with systemic administration of the NOP-selective non-peptide agonist Ro 64-6198 (Ko, M. C., et al., Neuropsychopharmacology, 2009, 34:2088-96). These anti-nociceptive effects of small-molecule NOP agonists are consistent with the anti-nociceptive effect of intrathecally administered N/OFQ peptide (see above) and confirmed in nonhuman primates (Ko, M. C., et al., J. Pharmacol Exp. Ther., 2006, 318:1257-64).

Since N/OFQ and NOP agonists modulate opioid anti-nociception and opioid-induced reward, Zaveri et al. hypothesized that dual-targeted, bifunctional NOP/mu opioid agonists may provide a novel approach for developing non-addicting analgesics (Toll, L., et al., J. Pharmacol Exp. Ther., 2009, 331:954-964). The Zaveri laboratory showed that the modestly selective NOP agonist 1-(1-cyclooctylpiperidin-4-yl)-indolin-2-one, which has mu opioid partial agonist activity, has anti-nociceptive activity in an acute pain measure, which is mediated by its mu-opioid activity and can be reversed by naloxone (Khroyan, T. V., et al., J. Pharmacol Exp. Ther., 2011, 339:687-693]), and also possesses potent anti-allodynic activity, mediated by its NOP agonist activity, which is reversible by the NOP antagonist SB-612111. Zaveri et al. also showed that 1-(1-cyclooctylpiperidin-4-yl)-indolin-2-one is not rewarding on its own, when evaluated in the CPP paradigm, and also has significant NOP-mediated anti-allodynic activity and is not rewarding on its own.

The NOP receptor agonism of the instant invention presents an excellent pharmacological strategy for providing pain relief in SCD, without the liabilities associated with opioid-based therapies, such as constipation, tolerance and dependence. The data with NOP/mu-opioid bifunctional agonists further suggests that such bifunctional compounds have a non-addicting profile and may have better efficacy in sickle cell pain, for long-term management of pain without dependence-related side effects.

### Chemical Compounds of the Invention

Small molecule nociceptin (NOP) receptor agonists and NOP receptor agonist/mu opioid receptor (MOR) partial agonists are known (Zaveri, N. T., Curr. Top. Med. Chem., 2011, 11:1151-1156). Such agonists are useful in the method of the present invention for the management of pain associated with sickle cell disease if they meet the selection criteria described below.

In one embodiment of the present invention, a series of indolinone derivatives, preferably piperidyl indolinones substituted on the nitrogen atom of the piperidinyl ring, are particularly suitable as either NOP receptor agonists or NOP receptor agonist/MOR partial agonists for use in the present invention. Such compounds are described in U.S. Published Application No. 2005/0228023 (Zaveri, et al.), “Agonist and Antagonist Ligands of the Nociceptin Receptor”, which is herein incorporated by reference in its entirety. In general, the indolinone derivatives have the structure of Formula I, or are pharmacologically active equivalents thereof:

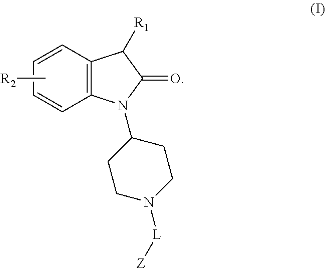

### In Formula I:

R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;

R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;

L is —(CHR³)_{P}—, —CH₂—Xᵥ—, or —C(═NH)—NR⁴;

p is an integer in the range of zero to 4 inclusive;

v is an integer in the range of 1 to 3 inclusive;

R³ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R³ may be the same or different;

R⁴ is selected from hydrogen, alkyl, cycloalkyl, cycloalkylalkyl, alkenyl, alkoxy, aryl and aralkyl;

X is independently CH₂, NR⁵ or 0, wherein at least one X, if v is greater than 1, is NR⁵ or O;

R⁵ is selected from hydrogen, alkyl, cycloalkyl, cycloalkylalkyl, alkenyl, alkoxy, aryl and aralkyl, and, in some preferred embodiments, when L is —(CHR³)_{P}—, p is 0; and

Z is hydrogen, cyclic hydrocarbyl, or arylcycloalkyl.

In another embodiment, Z is optionally substituted with one or more substituents selected from hydroxyl, halo, haloalkyl, amino, aminoalkyl, alkylamino, dialkylamino, alkyl, alkenyl, alkoxy, aryl, and aralkyl. In a further embodiment, Z has the structure

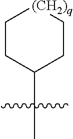

where q is an integer from 0-5.

The compounds of Formula I may be obtained as described in U.S. 2005/0228023. For example, [1-(1-cyclooctylpiperidin-4-yl)-indolin-2-one] is obtained by the condensation of the appropriately substituted benzyl amine with the appropriate N-substituted 4-piperidone, followed by decarboxylative cyclization, as described in Example 5 of U.S. 2005/0228023.

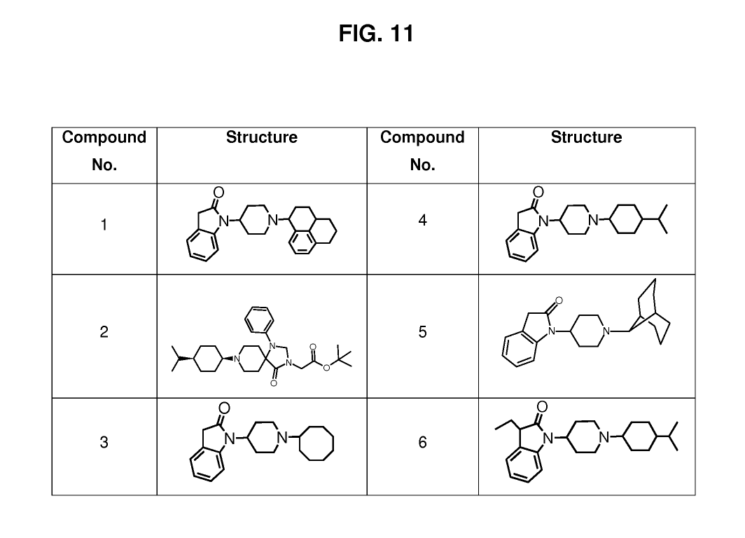

In one embodiment, compounds of Formula I include:
1-(1-(2,3,3a,4,5,6-hexahydro-1H-phenalen-1-yl)piperidinl-4-yl)-indolin-2-one (Compound 1);
1-(1-cyclooctylpiperidin-4-yl)-indolin-2-one (Compound 3; AT-200);
1-(1-(4-isopropylcyclohexyl)piperidin-4-yl)-indolin-2-one (Compound 4);
[1-(1-(Bicyclo[3.3.1]nonan-9-yl)piperidin-4-yl)indolin-2-one] (Compound 5); or
3-ethyl-1-(1-(4-isopropylcyclohexyl)piperidin-4-yl)-indolin-2-one (Compound 6).
   Structures for these compounds are illustrated in FIG. 11.

TABLE 1

| | Receptor Binding Kᵢ (nM) | | | [³⁵S]GTPyS NOP | | [³⁵S]GTPyS μ | | [³⁵S]GTPyS _{K} | |
|---|---|---|---|---|---|---|---|---|---|
| Cmpd | NOP | μ | K | EC₅₀ nM | % Stim | EC₅₀nM | % Stim | EC₅₀ nM | % Stim |

Selective (6-30 fold versus MOP) NOP Full Agonists

| | | | | | | | | | |
|---|---|---|---|---|---|---|---|---|---|
| 1 | 11.4 ± 0.9 | 79.8 ± 3.8 | 681 ± 61 | 46 ± 20 | 107 ± 7 | 129 ± 48 | 18 ± 2 | >10K | — |
| 2 | 0.97 ± 0.27 | 32.7 ± 13 | 270 ± 104 | 19 ± 11 | 81 ± 13 | 64 ± 26 | 29 ± 6 | 271 | 11 ± 2 |

Bifunctional NOP Agonist-Mu Opioid Agonists

| | | | | | | | | | |
|---|---|---|---|---|---|---|---|---|---|
| 3 | 1.39 ± 0.4 | 29.9 ± 2 | 42.6 ± 1 | 20 ± 3 | 54 ± 10 | 99 ± 12 | 23 ± 3.2 | 276 | 37 ± 3 |
| 4 | 3.96 ± 1.55 | 8.0 ± 0.97 | 148 ± 9 | 26 ± 4 | 100 ± 15 | 73 ± 5.3 | 49 ± 0.5 | >10K | — |
| 5 | 7.49 ± 0.8 | 2.7 ± 0.5 | 31.7 ± 4 | 28 ± 0.6 | 45 ± 5 | 30 ± 10 | 30 ± 0 | >10K | — |
| 6 | 5.22 ± 0.6 | 1.07 ± 0.2 | 82.4 ± 16 | 8.5 ± 0.8 | 95 ± 12 | 4.7 ± 1.1 | 44 ± 5.2 | >10K | — |

Table 1 shows the Kᵢ binding affinities and functional efficacies at the human NOP receptor for Compounds 1 and 3-6, in comparison with their activities at the δ and κ receptors. These data were obtained as described below in following sections.

The compounds of Formula I may be obtained as described in U.S. 2005/0228023. For example, [1-(1-cyclooctylpiperidin-4-yl)-indolin-2-one] is obtained by the condensation of the appropriately substituted benzyl amine with the appropriate N-substituted 4-piperidone, followed by decarboxylative cyclization, as described in Example 5 of U.S. 2005/0228023.

In a further embodiment, the nociceptin receptor agonist may be tert-butyl 2-{4-oxo-1-phenyl-8-[(1s,4s)-4-(propan-2-yl)cyclohexyl]-1,3,8-triazaspiro[4.5]decan-3-yl}acetate (Compound 2), which has a structure as shown in FIG. 11. Table 1 gives the Kᵢ binding affinity and its functional efficacy at the human NOP receptor for this NOP full agonist compound, in comparison with its activity at the δ and κ receptors.

In another embodiment of the present invention, the nociceptin receptor agonist may be ((1 S,3aS)-8-(2,3,3a,4,5,6-hexahydro-1H-phenalen-1-yl)-1-phenyl-1,3,8-triaza-spiro[4.5]decan-4-one), known as Ro64-6198, which has the structure shown below (7):

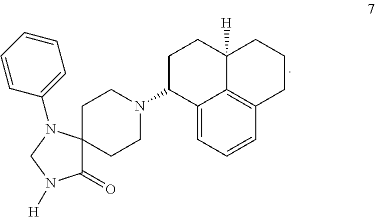

Reiss et al. have described the pharmacological activity of Ro64-6198 in vitro and in vivo in comparison to the mu receptor agonist morphine (Eur. J. Pharm., 2008, 579:141-148). Their study indicated that Ro64-6198 is an NOP receptor agonist as opposed to a mu receptor agonist. Compound 7 may be synthesized as described by Adam et al. (U.S. Pat. No. 6,071,925, issued Jun. 6, 2000).

In a further embodiment, the NOP receptor agonist may be 8-acenaphthen-1-yl-1-phenyl-1,3,8-triazaspiro(4.5)decan-4-one, known as Ro65-6570, which has the structure shown below (8):

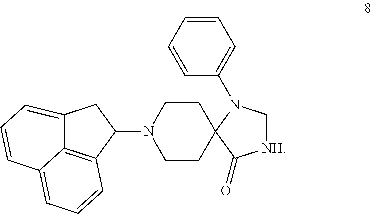 [SMILES: O=C1NCN(c2ccccc2)C12CCN(C1Cc3cccc4cccc1c34)CC2]

Rover et al. have described the biochemical characterization of this selective NOP receptor agonist (J. Med. Chem., 2000, 43:1329-1338) and Rutten et al. have reported on its in vivo effects on the acquisition of opiate- and psychostimulant-induced conditioned place preference in rats (Eur. J. Pharmacol., 2010, 645 (1-3):119-26). Compound 8 may be synthesized as described by Adam et al. (U.S. Pat. No. 6,071,925, issued Jun. 6, 2000).

In another embodiment, the NOP receptor agonist may be 1-[4-(propan-2-yl)cyclohexyl]-4-[3-(trifluoromethyl)phenyl]piperidin-4-ol, which has the structure shown below (9):

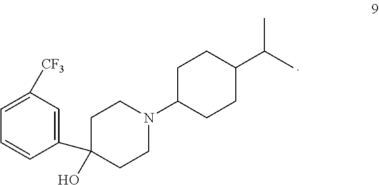 [SMILES: CC(C)C1CCC(N2CCC(O)(c3cccc(C(F)(F)F)c3)CC2)CC1]

Compound 9 may be prepared as described by Chen et al. (Bioorg. Med. Chem. Lett., 2003, 13:3247-3252). They also reported the Kᵢ binding affinity values shown in Table 2 for the compound as measured from binding to the human NOP receptor expressed in recombinant HEK-293 cells according to the method of Zhang et al. (J. Med. Chem., 2002, 45:5280).

TABLE 2

| NOP Kᵢ | mu Kᵢ | Selectivity | δ Kᵢ | Kappa Kᵢ |
|---|---|---|---|---|
| (nM) | (nM) | mu/NOP | (nM) | (nM) |
| 12 | 36 | 3.00 | 5674 | 4153 |

In further embodiment of the present invention, the nociceptin receptor agonist may be 1-[1-(1-methylcyclooctyl)-4-piperidinyl]-2-[(3R)-3-piperidinyl]-1H-benzimidazole, known as MCOPPB, which has the structure shown below (10):

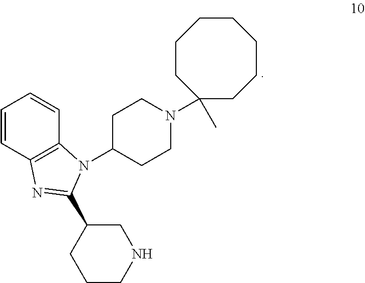 [SMILES: CC1(N2CCC(n3c([C@@H]4CCCNC4)nc4ccccc43)CC2)CCCCCCC1]

The compound may be prepared as described by Hayashi et al. (J. Med. Chem., 2009, 52(3):610-25) or as described by Ito et al. (Example 60, U.S. Pat. No. 6,172,067, issued Jan. 9, 2001), which is herein incorporated by reference in its entirety. MCOPPB is a high affinity NOP receptor agonist (pKᵢ=10.07 for the human NOP receptor), which exhibits anxiolytic effects with no effect on memory or locomotion. Hayashi et al. (2009) reported the binding affinity values for various opioid receptors shown in Table 3 as measured from binding to the human NOP receptors expressed in recombinant HEK-293 cells or CHO-K1 cells. Table 3 also gives EC₅₀ values for Compound 10 Compound as determined by the induction of the binding of [³⁵S]GTPγS to R-unit of G-protein due to binding of the compounds to hNOP receptors expressed in HEK-293 cells, human mu-receptors expressed in CHO-K1 cells, human κ-receptors expressed in HEK-293 cells, and human δ-receptors expressed in CHO-K1 cells. The Eₘₐₓ (efficacy) value was the maximal response calculated as the percentage of the maximal response produced by each control (N/OFQ, DAMGO, enadoline, and DPDPE).

TABLE 3

| | NOP | mu | δ | κ |
|---|---|---|---|---|
| Binding affinity Kᵢ | 0.0858 | 1.052 | 23.1 | >667 |
| (nM) | | | | |
| Functional activity | | | | |
| [³⁵S]GTPγS EC₅₀ | | | | |
| (nM) | 0.39 | 34 | 80 | 4696 |
| Eₘₐₓ (%) | 140% | 30% | 43% | 79% |

In another embodiment of the present invention, the nociceptin receptor agonist may be 8-[bis(2-methylphenyl)methyl]-3-phenyl-8-azabicyclo[3.2.1]octan-3-ol, known as SCH221510, which has the structure shown below (11):

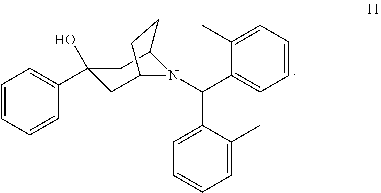 [SMILES: Cc1ccccc1C(c1ccccc1C)N1C2CCC1CC(O)(c1ccccc1)C2]

The compound may be prepared as described by Tulshian et al. (Table 7, U.S. Pat. No. 6,455,527, issued Sep. 24, 2002), which is herein incorporated by reference in its entirety. The compound is a potent piperidine NOP agonist (EC₅₀=12 nM) that binds with high affinity (Kᵢ=0.3 nM) and functional selectivity (>50-fold over the mu-, κ-, and δ-opioid receptors) as reported by Varty et al., (J. Pharm. Exper. Therap., 2008, 326(2):672-682).

In further embodiment of the present invention, the nociceptin receptor agonist may be endo-8-[bis(2-chlorophenyl)methyl]-3-phenyl-8-azabicyclo[3.2.1]octane-3-carboxamide, known as SCH655842, which has the structure shown below (12):

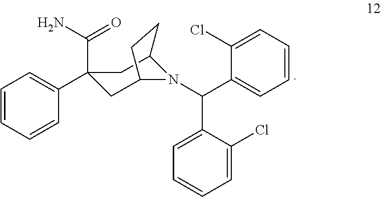 [SMILES: NC(=O)C1(c2ccccc2)CC2CCC(C1)N2C(c1ccccc1Cl)c1ccccc1Cl]

The compound may be prepared as described by Ho, et al., (Bioorg. Med. Chem. Lett., 2009, 19:2519-2523) and Tulshian et al. (U.S. Pat. No. 6,455,527, issued Sep. 24, 2002), which is herein incorporated by reference in its entirety. Ho, et al., (2009) further measured the in vitro and in vivo activities and pharmacokinetic profile for Compound 12. The compound is a potent NOP agonist, with a binding affinity Kᵢ of 1.7 nM at the NOP receptor, whereas its affinity for the other opioid receptors is Kᵢ=38 nM at the mu opioid receptor, Kᵢ=2326 nM at the δopioid receptor, and Kᵢ=268 nM at the κ opioid receptor. It has an EC₅₀ of 6 nM in the [³⁵S]GTPγS functional assay, for stimulation of the [³⁵S]GTPγS binding to the NOP receptor. Lu et al., reported a comparison with other opioids across rodent species of anxiolytic effects for Compound 12 (Eur. J. Pharm., 2011, 661:63-71).

In further embodiment of the present invention, the nociceptin receptor agonist may be (8-[bis(2-chlorophenyl)methyl]-3-(2-pyrimidinyl)-8-azabicyclo[3.2.1]octan-3-ol), known as SCH486757, which has the structure shown below (13):

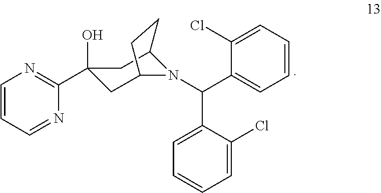 [SMILES: OC1(c2ncccn2)CC2CCC(C1)N2C(c1ccccc1Cl)c1ccccc1Cl]

The compound may be prepared as described by Ho, et al., (2009) and Tulshian et al. (Table 7, U.S. Pat. No. 6,455,527, issued Sep. 24, 2002), which is herein incorporated by reference in its entirety. McLeod, et al., reported that Compound 13 selectively binds human NOP receptor (Kᵢ=4.6±0.61 nM) over classical opioid receptors (Eur. J. Pharm., 2010, 630:112-120) with Table 4 showing the selectively of the compound against opioid receptors as determined by standard methods. The values in the table represent the mean±S.E.M. with the number of experiments shown in parentheses.

TABLE 4

| Receptor | Kᵢ (nM) | Fold Selectivity |
|---|---|---|
| NOP | 4.6 ± 0.61 (16) | 1 |
| mu | 972 ± 40 (4) | 211 |
| κ | 590 ± 40 (4) | 128 |
| δ | 14747 ± 2558 (4) | 3206 |

In a further embodiment of the present invention, the method for the management of pain associated with sickle cell disease, comprises administering to a patient in need of such pain management a therapeutically effective amount of a nociceptin (NOP) receptor agonist/mu opioid receptor (MOR) partial agonist, or a pharmaceutically acceptable salt, hydrate or solvate thereof. In one embodiment, the NOP/mu bifunctional compound may be buprenorphine (Compound 14), with the full chemical name [5α,7α(S)]-17-(cyclopropylmethyl)-α-(1,1-dimethylethyl)-4,5-epoxy-18,19-dihydro-3-hydroxy-6-methoxy-α-methyl-6,14-ethenomorphinan-7-methanol, hydrochloride, with the chemical structure:

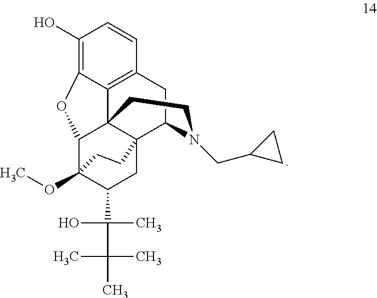

Buprenorphine is a clinically well-established opioid analgesic that shows complex interactions at the various opioid receptor subtypes. It shows high affinity to mu-, κ-, δ-, and nociceptin-opioid receptors with slow dissociation (Sadee et al., J. Pharmacol Exp. Ther., 1982, 223:157-162). Christoph et al., have published the results of experiments demonstrating the broad analgesic profile of buprenorphine in rodent models of acute and chronic pain (Eur. J. Pharmacol, 2005, 507 (1-3):87-98). Buprenorphine is commonly prescribed under the brand name Subutex® (Reckitt Benckiser Pharmaeuticals, Inc.; Richmond, Va.) and is used for the treatment of opioid dependence. Buprenorphine hydrochloride has also been recently approved by the U.S. Food & Drug Administration in a generic form for distribution by Amneal Pharmaceuticals, LLC (Bridgewater, N.J.).

In other embodiments, the NOP/mu bifunctional compound may be selected from those disclosed in U.S. Published Patent Application No. 2008/0125475, published May 29, 2008 (Linz, et al.), which is incorporated herein in its entirety. For example, the NOP/mu bifunctional compound may be (8-[bis(2-chlorophenyl)methyl]-3-(2-pyrimidinyl)-8-azabicyclo[3.2.1]octan-3-01), which has the structure shown below (15):

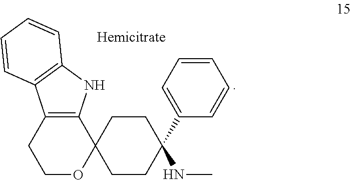

In another embodiment, the NOP/mu bifunctional compound may be 1,1-[3-dimethylamino-3-(2-thienyl)pentamethylene]-1,3,4,9-tetrahydropyrano[3,4-b]-6-fluoroindole hemicitrate, which has the structure shown below (16):

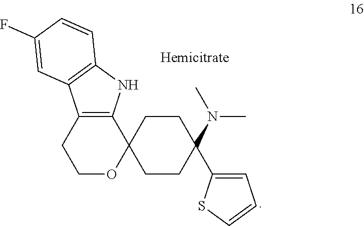

In a further embodiment, the NOP/mu bifunctional compound may be In another embodiment, the NOP/mu bifunctional compound may be 1,1-[3-dimethylamino-3-(3-thienyl)pentamethylene]-1,3,4,9-tetrahydropyrano[3,4-b]-6-fluoroindole, which has the structure shown below (17):

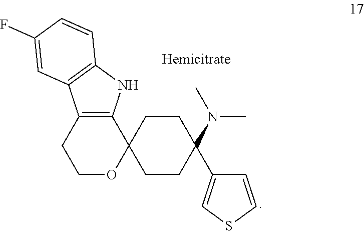

In a further embodiment, the NOP/mu bifunctional compound may be 1,1-[3-methylamino-3-(2-thienyl)pentamethylene]-1,3,4,9-tetrahydropyrano[3,4-b]indole, which has the structure shown below (18):

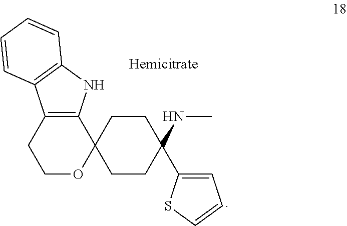

In another embodiment, the NOP/mu bifunctional compound may be N′-[2-(1H-indol-3-yl)-1-methyl-ethyl]-N,N-dimethyl-1-phenyl-cyclohexan-1,4-diamine citrate, which has the following structure (19):

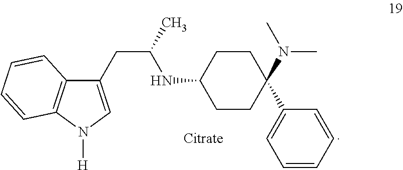 [SMILES: C[C@@H](Cc1c[nH]c2ccccc12)N[C@H]1CC[C@](c2ccccc2)(N(C)C)CC1]

These compounds may be prepared as described in U.S. Pat. No. 7,332,519, issued Feb. 19, 2008 (Hinze, et al.) or in U.S. 2008/0125475.

### Selection of NOP Receptor Monists and Bifunctional Monists

The criteria for selection of NOP receptor agonists suitable for use in the present invention include: 1) a high NOP binding affinity (Kᵢ) of less than about 50 nM); and 2) agonist efficacy of about 50% to about 100% of the natural NOP receptor agonist, nociceptin, as determined in a suitable assay that measures functional efficacy, such as in an GTP(γ)S assay.

Selection criteria for suitable bifunctional NOP agonist/mu agonists are: 1) NOP and mu binding affinity (Kᵢ) of less than about 50 nM; 2) NOP agonist efficacy equal to about 50 to about 100% of that of the natural NOP receptor agonist, nociceptin, as measured in a suitable assay that measures functional efficacy, such as an GTP(γ)S stimulation assay; and 3) mu efficacy equal to about 15% to about 75% of that of the peptide mu agonist DAMGO ([D-Ala2, N-MePhe4, Gly-ol]-enkephalin), a synthetic mu opioid peptide), as measured in a suitable functional efficacy assay such as GTP(γ)S stimulation.

### Measurement of Binding Affinity at the Human NOP Receptor

A suitable method for determining binding affinity at the human NOP receptor for use in the present invention involves a radioligand displacement assay. The binding affinity (Kᵢ nM) is determined using [³H]-nociceptin binding to membranes derived from Chinese Hamster Ovary (CHO) cells transfected with the human NOP receptor (also called the ORL1 receptor). ORL1-containing CHO cells are grown in Dulbecco's Modified Eagle Medium with 10% fetal bovine serum, in the presence of 0.4 mg/ml G418 and 0.1% penicillin/streptomycin, in 100-mm plastic culture dishes.

Binding to cell membranes is examined as described in Adapa et al. (Neuropeptides, 1997, 31:403-408). Cell membranes are resuspended in 50 mM Tris, pH 7.5, and the suspension is incubated with [³H]nociceptin/Orphanin FQ (N/OFQ), in a total volume of 1.0 ml, in a 96-well format, for 120 min at 25° C. Samples are filtered over glass fiber filters by using a Wallac cell harvester. Kᵢ values are determined by the curve-fitting program Prism, and are calculated from the formula Kᵢ=10₅₀/(1+L/Kd), where Kd is the binding affinity of [³H]N/OFQ and L is the concentration of [³H]N/OFQ used.

### Measurement of Functional Efficacy (Agonist Activity) at the Human NOP Receptor

For purposes of the present invention, functional efficacy at the human NOP receptor may be measured by a [³⁵5] GTPγS binding assay, conducted as described by Dooley, et al. (J. Pharmacol. Exp. Ther., 1997, 283:735-741). Cell membranes from human NOP receptor-transfected CHO cells are suspended in a buffer containing 20 mM HEPES, 10 mM MgCl₂, and 100 mM NaCl at pH 7.4. If needed, the cells are frozen at −70° C. prior to the final centrifugation. For the binding assay, membranes (10-20 mg protein) are incubated with [³⁵S]GTPγS (50 pM), GDP (usually 10 μM), and test compounds, in a total volume of 1 ml, for 60 min at 25° C. Samples are filtered over glass fiber filters and counted as described for the binding assays. A dose response with the NOP receptor natural peptide full agonist N/OFQ is conducted in each experiment to identify full and partial agonist compounds.

### Measurement of Binding Affinity at the Human Mu Opioid Receptor

A suitable method for determining binding affinity to the human μ opioid receptor for use in the present invention involves a radioligand displacement assay as described above for the human NOP receptor. [³H]DAMGO ([D-Ala2, N-MePhe4, Gly-ol]-enkephalin), is used as the radioligand.

### Measurement of Functional Efficacy (Agonist Activity) at the Human Mu Opioid Receptor

Functional efficacy at the mu opioid receptor may be measured by a [³⁵S] GTPγS binding assay, in a manner similar to that described above for the NOP receptor. A dose response with the mu receptor peptide full agonist DAMGO is conducted in each experiment to identify full and partial agonist compounds.

### Demonstration of Pain Modulation in Mammalian SCD Models

Compounds suitable for use in the method of the present invention are evaluated to determine the analgesic capabilities of NOP agonists and NOP agonist/mu partial agonists to modulate pain in transgenic SCD model mice. The effects studied are: 1) tonic hyperalgesia representing chronic pain; 2) acute pain due to vaso-occlusive crises incited by hypoxia/reoxygenation; and 3) inflammation and organ pathology to rule out the adverse effects of NOP-based analgesics.

Acute episodes of severe pain and chronic pain, ongoing inflammation and ischemia reperfusion injury that occur in SCD are also manifested in transgenic mice expressing sickle hemoglobin. In humans, and in experimental studies on mice, large doses of morphine are required to attenuate tonic hyperalgesia and complete Freund's adjuvant (CFA)-induced inflammatory pain. Mu opioid receptors are down-regulated in the skin and spinal cord of sickle mice and the clearance of morphine is higher in human patients with SCD as compared to normal subjects, supporting the requirement of high doses of morphine to treat pain in SCD.

To conduct experiments in SCD model mice, two types of sickle/control mice are bred, genotyped and phenotyped as described by Gupta and co-workers (Kohli, D. R. et al., Blood, 2010, 116:456-65). All experiments are performed on age- and sex-matched mice following institutional approvals. HbSSBERK (referred to as BERK mice) are homozygous for knockout of both murine α and β globins and carry a single copy of the linked transgenes for human α and β^{S} globins. Therefore, HbSS-BERK mice express human α and β^{S} globin chains with ˜99% human sickle hemoglobin, but no murine α or β globins (Paszty, C. et al., Science, 1997, 278:876-8). These mice exhibit cutaneous and deep tissue hyperalgesia (Kohli, 2010). BERK mice are from a mixed genetic background. Thus, litter-mate mixed genetic strain mice expressing normal human hemoglobin (HbAA-BERK) are used as a control for HbSS-BERK sickle mice.

The second type of mice utilized in SCD pain modeling experiments is S+S^{Antilles} mice. These mice are homozygous for the natural deletion of mouse β-major globin locus and transgenic expression of human α, β^{S}, and β^{S-Antilles} globin transgenes, with Antilles globin having a second mutation at β²³ position (valine to isoleucine) in addition to the β^{S} mutation on position β⁶ (Fabry, M. E., et al., Blood, 1995, 86:2419-2839). These mice express approximately 42% of human β^{S} and 36% of β^{S-Antilles} and show vascular pathology, inflammation and pain, which are further exacerbated by hypoxia/reoxygenation (Cain, D. M. et al., Brit. J. Haematol., 2012, 156(4):535-44; Belcher, J. D., et al., Blood, 2003, 101:3953-9; Kalambur, V. S., et al., Am. J. HematoL, 2004, 77:117-25). S+S^{Antilles} mice are on C57/BL6 background, which are used as controls.

Hypoxia/Reoxyqenation (H/R):

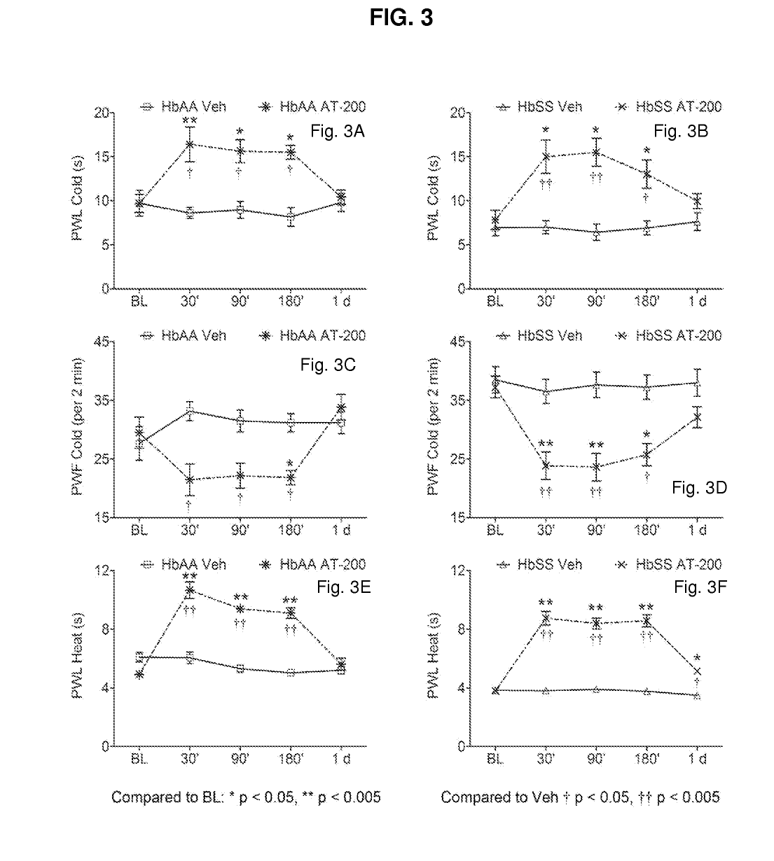

Following baseline pain measurements, HbSS-BERK and HbAA-BERK mice are exposed to hypoxia (H) with 8% O₂ and 92% N₂ for 3 h in a chamber maintained at 24° C., followed by re-oxygenation (R) with room air, as described by Gupta and co-workers (Cain, 2012). Pain measures will be obtained during reoxygenation, after 1-3 h and 24 h of re-oxygenation. Hypoxia induces approximately 10-fold increase in sickle RBC, stasis and reperfusion injury in sickle mice but not in normal mice, thus allowing the examination of pain due to vaso-occlusion similar to SCD crises. NOP agonist or NOP/MU agonists are injected subcutaneously at doses ranging from 0.3 to 10 mg/kg (based on affinity and data shown in FIG. 3), one hour after reoxygenation, followed by pain measures after 1 h, 2h, 4h and 24h.

Pain Behaviors.

All behavioral tests are performed after habituating the mice to each testing apparatus in a controlled environment prior to testing as described by Gupta and co-workers (Kohli, 2010). The tests performed are carefully ordered following post-hypoxia to allow sufficient acclimatization for in the mice before each test. The order followed is: 1) mechanical sensitivity; 2) sensitivity to heat; 3) grip force; and 4) cold sensitivity.

Withdrawal Responses to Mechanical Stimuli: Paw Withdrawal Threshold.

The mid-plantar surface of the hind paws are stimulated using von Frey monofilaments (Stoelting Co, Wood Dale, Ill.) with calibrated bending forces until the mouse withdraws its paw. Paw withdrawal threshold is determined using the updown paradigm of Chaplan et al. (Chaplan, S. R., et al., J. Neurosci. Methods, 1994, 53:55-63) modified by Hamamoto et al. (Hamamoto, D. T., et al., Eur. J. Pharmacol, 2007, 558:73-87) for mice. Testing is initiated with the 6.0 mN (0.61 g) von Frey filament. In the absence of a paw withdrawal to a given stimulus, a stronger stimulus is presented. In the case of a paw withdrawal, a weaker stimulus is presented. Six responses, starting with the negative response immediately before the first paw withdrawal, are recorded. The resulting pattern is tabulated and the 50% paw withdrawal threshold calculated using the formula: 50% g threshold=10_{f}^{(X+κδ)/}10,000; where X_{f}=value (in log units) of the final von Frey filament used; κ=tabular value for the pattern of positive/negative responses; and δ=mean difference (in log units) between stimuli (here, 0.223).

Paw Withdrawal Frequency (PWF) to Mechanical Stimulus.

The frequency (%) of paw withdrawal is obtained in response to 10 applications of a 9.8 mN (1.0 gram) filament standardized for sickle mice. The filament is applied for 1-2 s with an inter-stimulus interval of ˜5 s. Only vigorous withdrawals are recorded.

Withdrawal Responses to Heat.

Paw withdrawal latency (PWL) to a radiant heat source (Hargreaves test) is determined. Mice are placed on a temperature controlled (30° C.) glass platform, and radiant heat is applied to the middle of the plantar surface of the hind paw and PWL is determined, for 3 trials for each hind paw, with each trial separated by at least 5 min. The intensity of heat source is adjusted so that at baseline, mice withdraw their hind paws at ˜9s and a cut-off time of 16s is chosen to avoid tissue damage.

Grip Force:

For deep tissue hyperalgesia, the tensile force of peak forelimb exertion is measured using a computerized grip force meter. Each mouse is held by its tail and gently passed over a wire mesh grid and allowed to grip the wires. The peak force exerted against the transducer is recorded in grams.

Withdrawal Responses to Cold:

Mice are placed on a cold plate (4° C.) and the latency (s) to initial lifting of either forepaw (paw withdrawal latency), and the number of forepaw withdrawals (paw withdrawal frequency) during a period of 2 minutes on the cold plate is recorded.

Statistical Analyses.

Multiple comparisons after analysis of variance are performed using a Tukey's test. For hypoxia/reoxygenation results, all data are compared first by two-way analysis of variance for between-group comparisons over time and multiple comparisons using Tukey's post hoc testing. For within group comparisons over time, all data are compared using one-way analysis of variance and multiple comparisons after analysis are completed using Tukey's post hoc testing. A P value of less than 0.05 is considered significant. Data are analyzed using Prism software (GraphPad, San Diego, Calif.).

### Pharmaceutical Compositions

The present invention provides compositions (including pharmaceutical compositions) comprising an effective amount of one or more NOP receptor agonists and/or NOP receptor agonist/mu opioid receptor partial agonists as described herein and a suitable diluent, physiologically acceptable excipient, or carrier. The agonists administered in vivo preferably are in the form of a pharmaceutical composition.

The compositions of the present invention may contain the agonists in any form described herein. Agonists of the invention may be formulated according to known methods that are used to prepare pharmaceutically useful compositions. Components that are commonly employed in pharmaceutical formulations include those described in Remington: The Science and Practice of Pharmacy (21^{st} Edition, University of the Sciences in Philadelphia, 2005)

The agonists employed in a pharmaceutical composition preferably are purified such that the agonist is substantially free of other compounds of natural or endogenous origin, desirably containing less than about 1% purity by mass of contaminants residual of production processes. Such compositions, however, can contain other materials added as stabilizers, carriers, excipients or co-therapeutics.

For therapeutic use, the compositions are administered in a manner and dosage appropriate to the desired effect, indication and the patient. Administration may be by any suitable route, including but not limited to, oral, nasal, buccal, continuous infusion, local administration, sustained release from implants (gels, membranes, and the like), intravenous injection, subcutaneous or intramuscular injection.

The compositions of the present invention may be administered in conjunction with other therapeutic modalities known in the art as treatments for SCD and its complications, including, but not limited to, other pain medications, antibiotics, antivirals, and hydroxyurea. In addition, the compositions find use during treatment of sickle cell crises and during administration of rehydrating fluids and oxygen therapy. The compositions may also be administered in conjunction with blood transfusions used to treat anemia, or with blood and marrow stem cell transplants.

### EXAMPLES

### Example 1

### Demonstration of Pain Modulation in SCD Models

Pain-related behaviors approaches were used to investigate the modulation of pain response by the NOP receptor system in HbSS-BERK mice using the NOP receptor agonist, AT-200 (Compound 3). Changes in cutaneous skin temperature and blood flow that are indicative of inflammatory state were also compared in these models.

NOP Receptor Agonist AT-200 Ameliorates Mechanical and Deep Tissue Hyperalgesia.

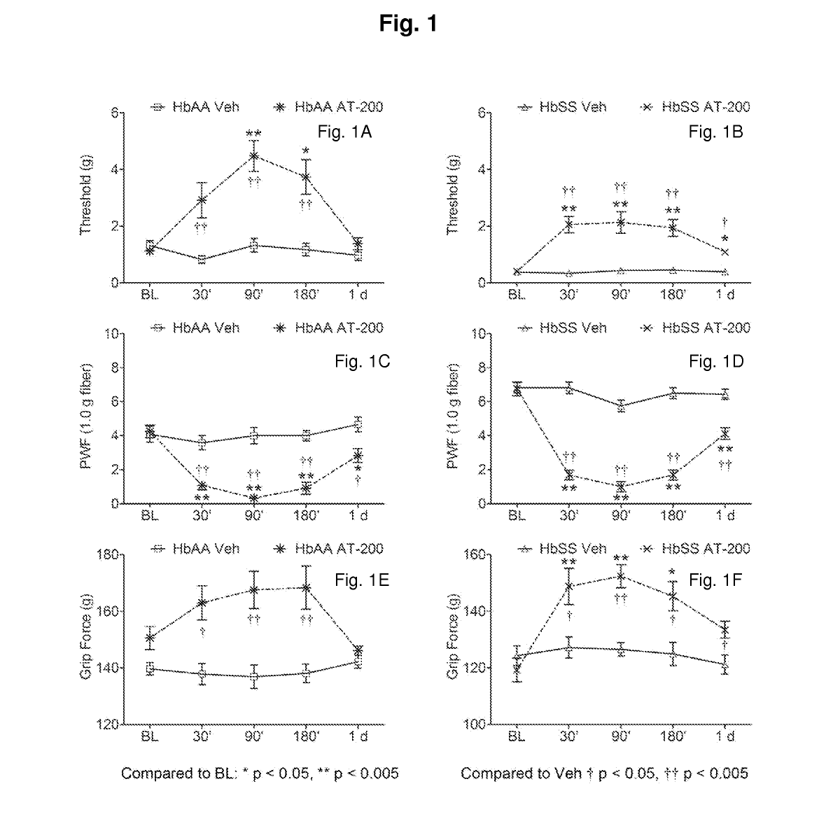

The mechanical withdrawal threshold obtained using von Frey monofilaments increased after 30 min of injection of AT-200 and remained increased after 24 h (last period of observation) as compared to baseline observations in the sickle HbSS-BERK mice (FIG. 1B). An increase in withdrawal threshold occurred in HbAA-BERK following 90 min of injection, but returned to baseline after 24 h (FIG. 1A). In contrast, the withdrawal threshold did not increase in vehicle-treated mice following injection at any time point. The withdrawal threshold remained significantly increased in AT-200-treated mice as compared to vehicle-treated mice up to 24 h and 180 min in HbSS-BERK and HbAA-BERK mice, respectively.

Paw withdrawal frequency (PWF) evoked by the standard 9.8 mN (1.0 g) von Frey monofilament decreased after 30 min of injection and remained decreased after 24 h as compared to their baseline in both HbSS-BERK and HbAA-BERK mice (FIGS. 1C and 1D). In contrast, PWF did not change in vehicle-treated mice following injection at any time point. PWF remained significantly decreased in AT-200-treated mice as compared to vehicle-treated mice up to 24 h after injection in both HbSS-BERK and HbAA-BERK mice.

Grip force increased after 30 min of injection and remained increased until 180 min in HbSS-BERK mice as compared to baseline, but not in the control HbAA-BERK (FIGS. 1E and F). In contrast, grip force did not increase in vehicle-treated mice following injection at any time point. Grip force remained significantly increased in AT-200-treated mice as compared to vehicle-treated mice up to 180 min in both mice. A lower force indicates increased nociception, the neural processes of encoding and processing noxious stimuli. The significant sustained decrease in mechanical sensitivity parameters from 30 min to 24 h and increase in grip force from 30 min to 180 min after AT-200 treatment suggests that the NOP receptor agonist ameliorates pain in HbSS-BERK mice. It is noteworthy that HbSS-BERK mice demonstrated greater hyperalgesia in all three parameters than HbAA-BERK mice.

NOP Receptor Agonist AT-200 Ameliorates Mechanical and Deep Tissue Hyperalgesia Following Hypoxia/Reoxygenation Injury.

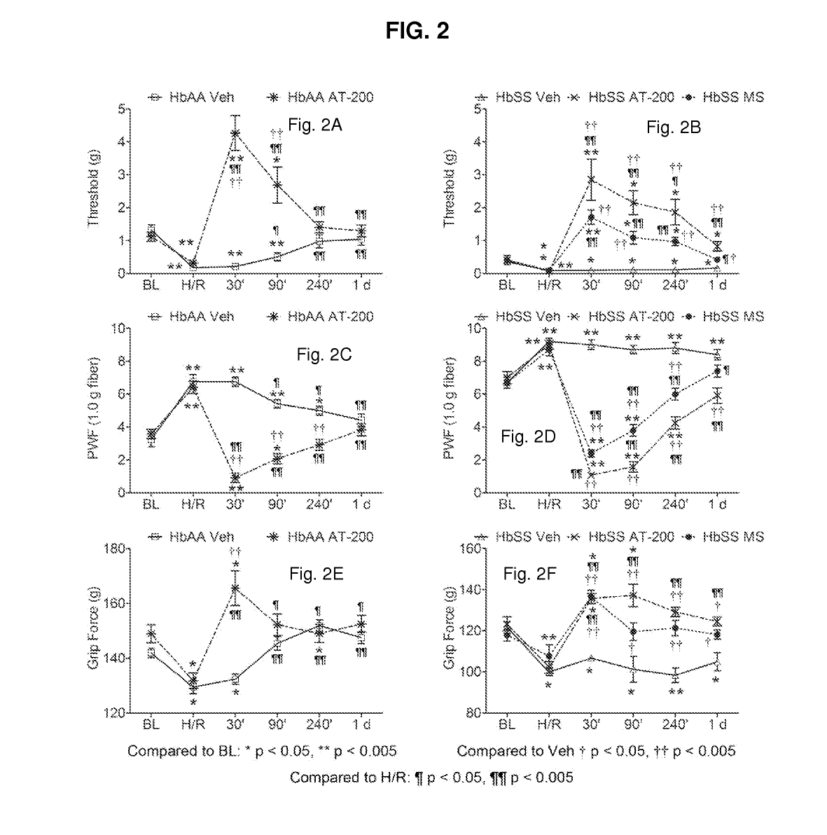

HbSS-BERK mice injected with AT-200 following hypoxia/reoxygenation (H/R) injury, showed a significant increase in mechanical withdrawal threshold obtained using von Frey monofilaments after 30 min of injection and remained increased after 24 h (last period of observation) as compared to their values at baseline and immediately after H/R injury in the sickle HbSS-BERK mice (FIG. 2B). The withdrawal threshold continued to remain higher up to 24 h in HbSS-BERK mice injected with morphine following H/R injury and returned to baseline at this time point. In contrast, the withdrawal threshold did not increase in vehicle-treated HbSS-BERK mice following H/R injury at any time point. The withdrawal threshold remained significantly decreased in vehicle-treated mice up to 24 h as compared to baseline (before H/R). The withdrawal threshold remained significantly increased in AT-200-treated mice and morphine-treated mice as compared to vehicle-treated mice up to 24 h in HbSS-BERK, but only up to 90 min in HbAA-BERK mice. An increase in withdrawal threshold occurred in HbAA-BERK following 30 min of injection, but returned to baseline after 240 min (FIG. 2A). The withdrawal threshold returned to baseline following 240 min of injection in vehicle-treated HbAA-BERK mice following H/R. The withdrawal threshold remained significantly increased in AT-200-treated HbAA-BERK mice as compared to vehicle-treated mice up to 90 min.

PWF evoked by the 9.8 mN (1.0 g) von Frey monofilament significantly decreased after 30 min of injection of Compound 3 and remained decreased after 24 h as compared to the PWF at baseline and immediately after H/R in HbSS-BERK (FIG. 2D). Similarly, the PWF in HbSS-BERK mice injected with morphine continued to remain lower up to 24 h as compared to immediately after H/R, but returned to baseline at this time point. In contrast, PWF did not decrease in vehicle-treated HbSS-BERK mice following H/R at any time point. PWF remained significantly increased in vehicle-treated mice up to 24 h as compared to baseline in HbSS-BERK. A decrease in PWF occurred at 30 min as compared to immediately after H/R in AT-200 treated HbAA-BERK mice (FIG. 2C). PWF remained lower up to 24 h as compared to H/R and returned to baseline at 240 minutes. PWF significantly decreased after 90 min of H/R in vehicle-treated HbAA-BERK mice as compared to immediately after H/R, but remained increased up to 240 min as compared to baseline.

HbSS-BERK mice injected with AT-200 following H/R showed a significant increase in grip force after 30 min of injection as compared to the grip force immediately after H/R (FIG. 2F). Grip force remained increased in AT-200-treated mice up to 24 h and returned to baseline at 240 min of injection. Grip force in morphine-treated HbSS-BERK mice significantly increased only after 30 min of injection as compared to baseline and immediately after H/R, but returned to baseline at 90 minutes. In contrast, vehicle-treated mice grip force did not increase following H/R at any time point. Grip force remained significantly increased in AT-200-treated and morphine-treated HbSS-BERK mice as compared to vehicle-treated mice up to 24 h. HbAA-BERK mice injected with AT-200 following H/R showed a significant increase in grip force at 30 min and remained increased up to 24 h as compared to immediately after H/R (FIG. 2E). However, grip force returned to baseline at 90 min in both HbAA-BERK treatment groups. These data demonstrate that sickling, vaso-occlusion, and ischemia-reperfusion injury incited by H/R injury possibly lead to increased and sustained mechanical sensitivity and deep tissue/musculoskeletal pain in sickle mice. Control mice lacking sickle hemoglobin do not show sustained mechanical sensitivity and deep tissue pain with pain parameters returning to baseline levels at varying time points. A sustained increase in withdrawal threshold, decrease in PWF, and an increase in grip force from 30 min to 24 h after AT-200 treatment suggests that NOP receptor agonist ameliorates mechanical sensitivity and deep tissue pain incited by H/R injury in HbSS-BERK mice.

NOP Receptor Aqonist AT-200 Ameliorates Thermal Sensitivity.

HbSS-BERK and their control HbAA-BERK mice exhibited a significant decrease in cold sensitivity following AT-200 treatment from 30 min to 180 min (FIGS. 3A-D). Paw withdrawal latency (PWL) and PWF to cold in both mice returned to baseline at 24 h of treatment. Sustained decrease in sensitivity to heat was observed in HbSS-BERK from 30 min to 24 h but only to 180 min in HbAA-BERK (FIGS. 3E and 3F). In contrast, thermal sensitivity did not change in vehicle-treated mice following treatment. Thus, these data demonstrated that HbSS-BERK mice have enhanced cold and heat sensitivity.

NOP Receptor Agonist AT-200 Ameliorates Thermal Sensitivity Following Hypoxia/Reoxygenation Injury.

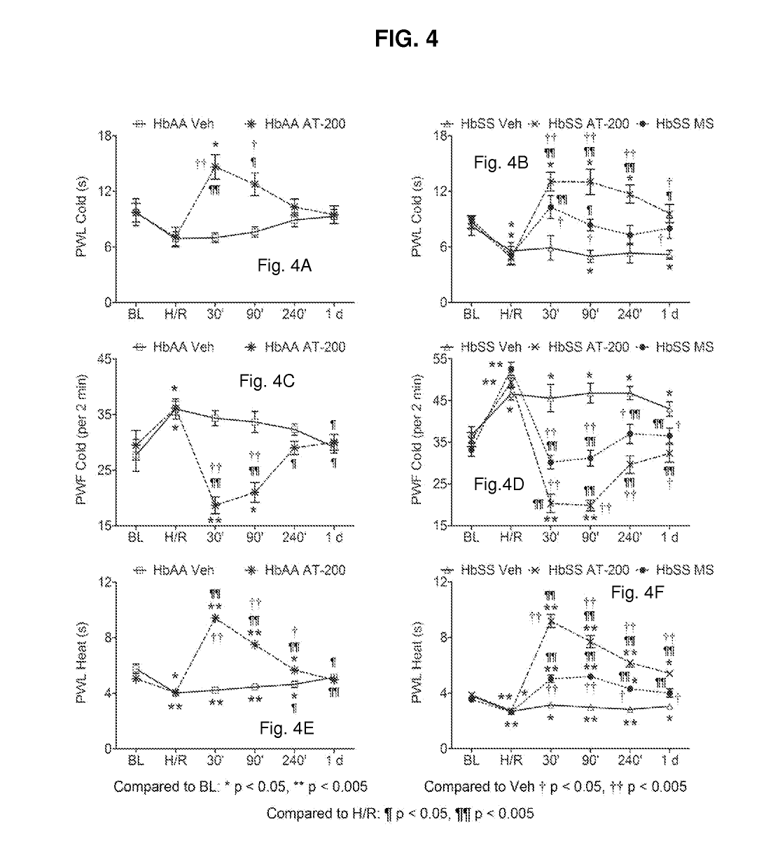

HbSS-BERK mice injected with AT-200 following H/R injury showed a significant decrease in cold sensitivity at 30 min of injection as compared to immediately after H/R injury (FIGS. 4B and 4D). HbSS-BERK mice treated with AT-200 showed a sustained increase in PWL to cold and decrease in PWF to cold up to 24 h as compared to immediately after H/R and returned to baseline at 240 min. Morphine-treated HbSS-BERK mice showed a significant increase in PWL to cold from 30 min to 90 min of injection and a significant decrease in PWF to cold from 30 min to 24 h as compared to immediately after H/R. It is noteworthy that in AT-200-treated HbSS-BERK mice, PWL and PWF to cold returned to baseline at 240 min and 90 min, respectively, but in morphine-treated HbSS-BERK mice, these measurements returned to baseline at 30 min in both cold tests. In contrast, vehicle-treated HbSS-BERK mice PWL to cold remained decreased after 24 h following H/R and the PWF to cold did not decrease following H/R injury at any time point. HbAA-BERK treated with AT-200 following H/R, showed a significant increase in PWL to cold from 30 min to 90 min and decrease in PWF to cold from 30 min to 24 h as compared to immediately after H/R (FIGS. 4A and 4C). PWL and PWF to cold returned to baseline at 90 min and 240 min, respectively, in the AT-200-treated HbAA-BERK mice. In contrast, PWL to cold did not change from baseline values in vehicle-treated mice following H/R or treatment. PWF to cold significantly increased immediately after H/R as compared to baseline and returned to baseline at 30 min of treatment.

HbSS-BERK and HbAA-BERK mice exhibited a significant increase in PWL to heat following AT-200 treatment from 30 min to 24 h as compared to immediately after H/R (FIGS. 4E and 4F). PWL to heat remained increased up to 24 h as compared to baseline in HbSS-BERK mice treated with AT-200, but returned to baseline at 24 h in morphine-treated HbSS-BERK mice and Compound 3 HbAA-BERK mice. In contrast, PWL to heat did not increase in vehicle-treated HbSS-BERK mice following H/R at any time point, but HbAA-BERK mice increased at 240 min. PWL to heat remained significantly decreased in vehicle-treated HbSS-BERK and HbAA-BERK mice up to 24 h and 240 min as compared to baseline, respectively. These data demonstrate the sustained sensitivity to cold and heat incited by H/R. Therefore, a significant sustained increase to PWL to cold and heat and decrease to PWF to cold from 30 min to 24 h after AT-200 treatment suggests that the NOP receptor agonist ameliorates thermal sensitivity incited by H/R injury in HbSS-BERK mice.

NOP Receptor Agonist AT-200 does not Affect Dorsal Cutaneous Blood Flow.

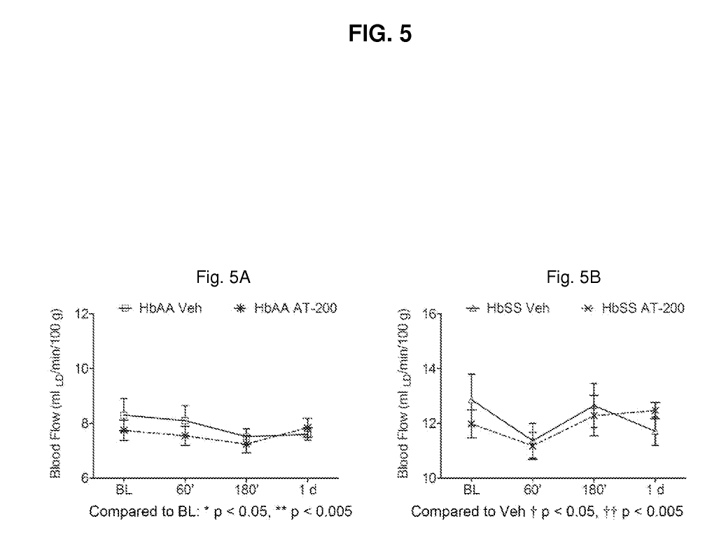

Neither HbSS-BERK nor HbAA-BERK mice showed any changes to dorsal cutaneous blood flow at any time point following AT-200 treatment (FIG. 5). It is noteworthy that HbSS-BERK mice demonstrated an increased blood flow rate.

NOP Receptor Agonist AT-200 Reduces Dorsal Cutaneous Blood Flow Following Hypoxia/Reoxygenation Injury.

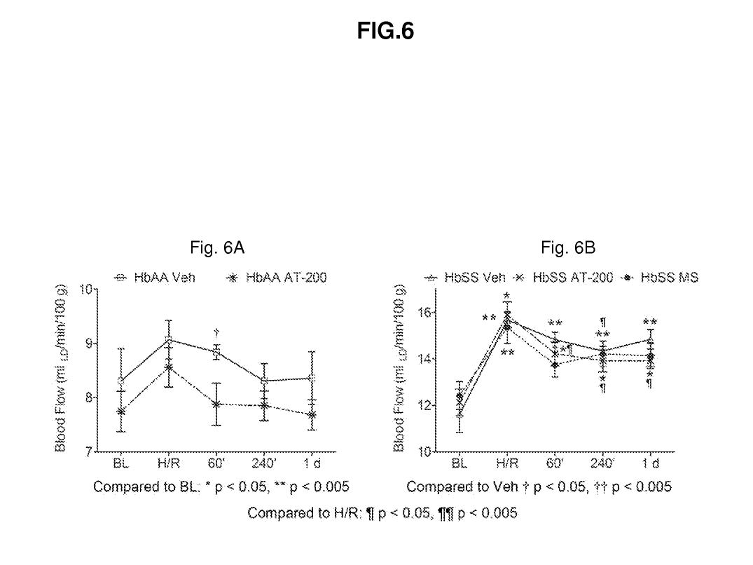

HbSS-BERK mice exhibited a significant increase in dorsal cutaneous blood flow following H/R injury as compared to baseline (FIG. 6B). AT-200-treated HbSS-BERK mice showed a significant decrease in blood flow after 60 min to 24 h after injection as compared to blood flow immediately after H/R injury, but did not return to baseline at any time point. In contrast, morphine- and vehicle-treated HbSS-BERK mice did not decrease in blood flow following H/R injury at any time point, but morphine-treated HbSS-BERK mice returned to baseline after 30 min injection. However, neither HbAA-BERK treatment group exhibited an increased in blood flow (FIG. 6A). These data demonstrate that sickling, vaso-occlusion and ischemia-reperfusion injury induced by H/R injury possibly leads to increased dorsal cutaneous blood flow since the control mice do not show any change in blood flow rate.

NOP Receptor Agonist AT-200 Reduces Dorsal Skin Temperature.

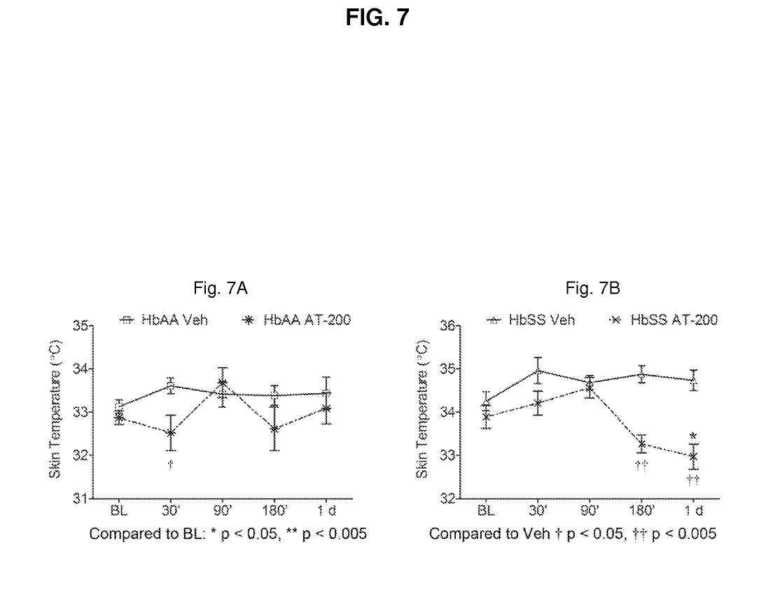

HbSS-BERK mice injected with AT-200 showed a significant decrease in dorsal skin temperature 24 h after injection as compared to skin temperature measured at baseline (FIG. 7B). In contrast, skin temperature did not change in vehicle-treated mice following treatment. Neither treatment group in the HbAA-BERK mice showed any changes to dorsal skin temperature at any time point (FIG. 7A).

NOP Receptor Agonist AT-200 Reduces Dorsal Skin Temperature Following Hypoxia/Reoxygenation.

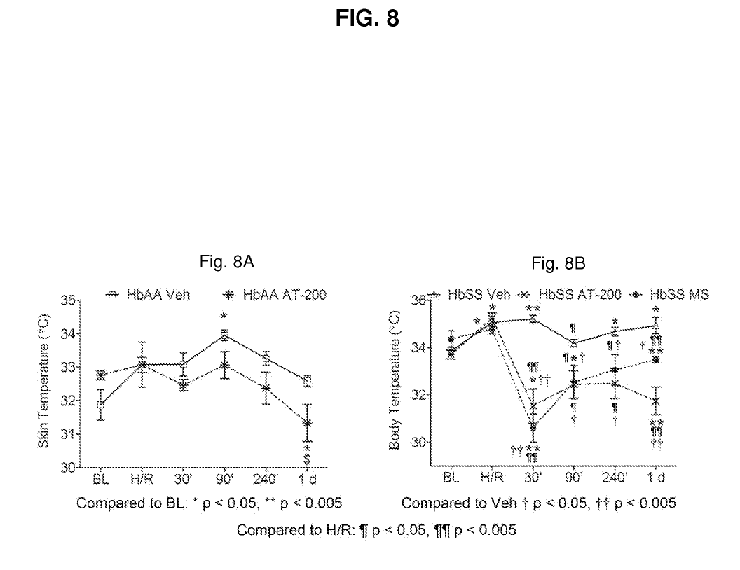

HbSS-BERK mice exhibited a significant increase in dorsal skin temperature following H/R injury as compared to baseline (FIG. 8B). AT-200- and morphine-treated HbSS-BERK mice showed a significant decrease in skin temperature from 30 min to 24 h after treatment as compared to skin temperature immediately after H/R. Skin temperature returned to baseline at 90 min but significantly decreased after 24 h for AT-200- and morphine-treated HbSS-BERK mice. In contrast, vehicle-treated HbSS-BERK mice did not decrease in skin temperature up to 24 h following H/R, except at 90 min. HbAA-BERK mice treated with AT-200 did not exhibit any changes in skin temperature as compared to baseline and immediately after H/R injury, except for at 24 h (FIG. 8A). In contrast, vehicle-treated HbAA-BERK mice only exhibited increased skin temperature at 90 min and remained at baseline values for all other time points. These data demonstrate that the inflammatory responses due to sickling, vaso-occlusion and ischemia-reperfusion injury induced by H/R injury possibly leads to increased dorsal skin temperature, since control mice do not show any change to skin temperature.

Importantly, mice injected with AT-200 did not exhibit catalepsy. There was no significant difference in the time spent on the bar between AT-200- and vehicle-treated mice at any time points. Thus, the anti-nociceptive effects of AT-200 were not due to impaired motor functions.

All publications and patent applications mentioned in this specification are herein incorporated by reference to the same extent as if each individual publication or patent application were specifically and individually indicated to be incorporated by reference.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for the management of pain associated with sickle cell disease, comprising administering to a patient in need of such pain management a therapeutically effective amount of a nociceptin (NOP) receptor agonist, or a pharmaceutically acceptable salt, hydrate or solvate thereof.

2. The method according to claim 1, wherein the pain management comprises preventing or treating said pain.

3. The method according to claim 1, wherein the administering occurs during or after a sickle cell crisis.

4. The method according to claim 1, wherein the NOP receptor agonist exhibits an NOP binding affinity of less than about 50 nM) and an agonist efficacy of about 50% to about 100% of nociceptin.

5. The method according to claim 4, wherein the agonist efficacy is determined in a GTP(γ)S assay.

6. The method according to claim 4, wherein the NOP receptor agonist comprises a compound having the structure of Formula 1: 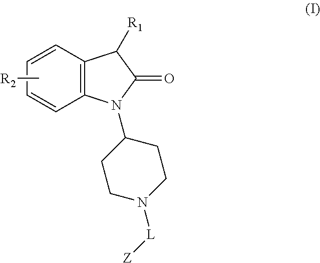
wherein:
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
L is —(CHR³)_{P}—, —CH₂—Xᵥ—, or —C(═NH)—NR⁴;
p is an integer in the range of zero to 4 inclusive;
v is an integer in the range of 1 to 3 inclusive;
R³ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R³ may be the same or different;
R⁴ is selected from hydrogen, alkyl, cycloalkyl, cycloalkylalkyl, alkenyl, alkoxy, aryl and aralkyl;
X is independently CH₂, NR⁵ or O, wherein at least one X, if v is greater than 1, is NR⁵ or O;
R⁵ is selected from hydrogen, alkyl, cycloalkyl, cycloalkylalkyl, alkenyl, alkoxy, aryl and aralkyl, and, in some preferred embodiments, when L is —(CHR³)P—, p is 0; and
Z is hydrogen, cyclic hydrocarbyl, or arylcycloalkyl.

7. The method according to claim 6, wherein the NOP receptor agonist is 1-(1-(2,3,3a,4,5,6-hexahydro-1H-phenalen-1-yl)piperidinl-4-yl)-indolin-2-one.

8. The method according to claim 6, wherein Z has the structure: 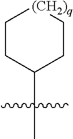
where q is an integer from 0-5.

9. The method according to claim 8, wherein the NOP receptor agonist is 1-(1-cyclooctylpiperidin-4-yl)-indolin-2-one.

10. The method according to claim 1, wherein the NOP receptor agonist is:
tert-butyl 2-{4-oxo-1-phenyl-8-[(1s,4s)-4-(propan-2-yl)cyclohexyl]-1,3,8-triazaspiro[4.5]decan-3-yl}acetate;
((1S,3aS)-8-(2,3,3a,4,5,6-hexahydro-1H-phenalen-1-yl)-1-phenyl-1,3,8-triaza-spiro[4.5]decan-4-one);
8-acenaphthen-1-yl-1-phenyl-1,3,8-triazaspiro(4.5)decan-4-one;
1-[4-(propan-2-yl)cyclohexyl]-4-[3-(trifluoromethyl)phenyl]piperidin-4-ol;
1-[1-(1-methylcyclooctyl)-4-piperidinyl]-2-[(3R)-3-piperidinyl]-1H-benzimidazole;
8-[bis(2-methylphenyl)methyl]-3-phenyl-8-azabicyclo[3.2.1]octan-3-ol;
endo-8-[bis(2-chlorophenyl)methyl]-3-phenyl-8-azabicyclo[3.2.1]octane-3-carboxamide;
or
(8-[bis(2-chlorophenyl)methyl]-3-(2-pyrimidinyl)-8-azabicyclo[3.2.1]octan-3-ol).

11. A method for the management of pain associated with sickle cell disease, comprising administering to a patient in need of such pain management a therapeutically effective amount of a nociceptin (NOP) receptor agonist/mu opioid receptor (MOR) partial agonist, or a pharmaceutically acceptable salt, hydrate or solvate thereof.

12. The method according to claim 11, wherein the pain management comprises preventing or treating said pain.

13. The method according to claim 11, wherein the administering occurs during or after a sickle cell crisis.

14. The method according to claim 11, wherein the NOP receptor agonist/MOR partial agonist exhibits an NOP and mu binding affinity (Kᵢ) of less than about 50 nM, an NOP agonist efficacy equal to about 50 to about 100% of that of nociceptin, and an mu efficacy equal to about 15% to about 75% of that of the peptide mu agonist DAMGO ([D-Ala2, N-MePhe4, Gly-ol]-enkephalin).

15. The method according to claim 14, wherein the NOP agonist efficacy is determined in an GTP(γ)S assay.

16. The method according to claim 14, wherein the mu efficacy is determined in an GTP(γ)S assay.

17. The method according to claim 14, wherein the NOP receptor agonist/MOR partial agonist comprises a compound having the structure of Formula 1: 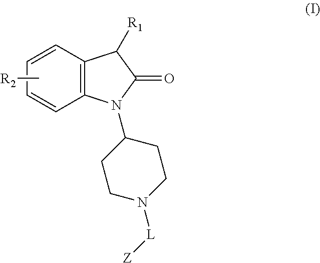
wherein:
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
L is —(CHR³)_{P}—, —CH₂—Xᵥ—, or —C(═NH)—NR⁴;
p is an integer in the range of zero to 4 inclusive;
v is an integer in the range of 1 to 3 inclusive;
R³ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R³ may be the same or different;
R⁴ is selected from hydrogen, alkyl, cycloalkyl, cycloalkylalkyl, alkenyl, alkoxy, aryl and aralkyl;
X is independently CH₂, NR⁵ or O, wherein at least one X, if v is greater than 1, is NR⁵ or O;
R⁵ is selected from hydrogen, alkyl, cycloalkyl, cycloalkylalkyl, alkenyl, alkoxy, aryl and aralkyl, and, in some preferred embodiments, when L is —(CHR³)_{P}—, p is 0; and
Z is hydrogen, cyclic hydrocarbyl, or arylcycloalkyl.

18. The method according to claim 17, wherein the NOP receptor agonist/MOR partial agonist is:
1-(1-cyclooctylpiperidin-4-yl)-indolin-2-one;
1-(1-(4-isopropylcyclohexyl)piperidin-4-yl)-indolin-2-one;
[1-(1-(Bicyclo[3.3.1]nonan-9-yl)piperidin-4-yl)indolin-2-one]; or
3-ethyl-1-(1-(4-isopropylcyclohexyl)piperidin-4-yl)-indolin-2-one.

19. The method according to claim 17, wherein Z has the structure: 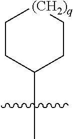
where q is an integer from 0-5.

20. The method according to claim 19, wherein the NOP receptor agonist/MOR partial agonist is 1-(1-cyclooctylpiperidin-4-yl)-indolin-2-one.

21. The method according to claim 11, wherein the NOP receptor agonist/MOR partial agonist is:
[5α,7α(S)]-17-(cyclopropylmethyl)-α-(1,1-dimethylethyl)-4,5-epoxy-18,19-dihydro-3-hydroxy-6-methoxy-α-methyl-6,14-ethenomorphinan-7-methanol, hydrochloride;
8-[bis(2-chlorophenyl)methyl]-3-(2-pyrimidinyl)-8-azabicyclo[3.2.1]octan-3-ol);
1,1-[3-dimethylamino-3-(3-thienyl)pentamethylene]-1,3,4,9-tetrahydropyrano[3,4-b]-6-fluoro indole;
1,1-[3-methylamino-3-(2-thienyl)pentamethylene]-1,3,4,9-tetrahydropyrano[3,4-b]indole;
or
N′-[2-(1H-indol-3-yl)-1-methyl-ethyl]-N,N-dimethyl-1-phenyl-cyclohexan-1,4-diamine citrate.

22. A pharmaceutical composition for managing pain associated with sickle cell disease, comprising a therapeutically effective amount of an NOP agonist or an NOP agonist/mu opioid partial agonist, or a pharmaceutically acceptable salt, hydrate or solvate thereof, and a pharmaceutically acceptable carrier.
